(19) United States
(12) Patent Application Publication
(10) Pub. No.: US 2026/0139274 A1
(43) Pub. Date: May 21, 2026

(54) GENE THERAPY FOR JUVENILE BATTEN DISEASE

(71) Applicants: BOARD OF REGENTS OF THE UNIVERSITY OF NEBRASKA, Omaha, NE (US); OHIO STATE INNOVATION FOUNDATION, Columbus, OH (US)

(72) Inventors: Tammy Kielian, La Vista, NE (US); Kevin Foust, New Albany, OH (US)

(73) Assignees: BOARD OF REGENTS OF THE UNIVERSITY OF NEBRASKA, Omaha, NE (US); OHIO STATE INNOVATION FOUNDATION, Columbus, OH (US)

(21) Appl. No.: 19/183,295

(22) Filed: Apr. 18, 2025

Related U.S. Application Data
(63) Continuation of application No. 17/131,564, filed on Dec. 22, 2020, now Pat. No. 12,297,447.
Application No. 17/131,564 is a continuation of application No. 15/537,384, now Pat. No. 10,876,134.
(60) Provisional application No. 62/092,501, filed on Dec. 16, 2014.
(60) Provisional application No. 62/146,793, filed on Apr. 13, 2015.

(51) Int. Cl. C12N 15/864; A61K 48/00; A61P 25/00; C12N 15/86

(52) U.S. Cl. CPC C12N 15/8645; A61K 48/00; A61P 25/00; C12N 15/86; C12N 2750/14143; C12N 2750/14171; C12N 2830/008

(57) ABSTRACT

Compositions and methods for the treatment of Juvenile Neuronal Ceroid Lipofuscinosis (JNCL), also known as Juvenile Batten Disease, are provided herein. In certain embodiments the compositions include but are not limited to adeno-associated viral (AAV) constructs, including self-complementary adeno-associated viral (sc-AAV) constructs, that express the human gene CLN3 (or a CLN3 cDNA).

### CROSS-REFERENCE TO RELATED APPLICATIONS

This application is a Continuation of U.S. patent application Ser. No. 17/131,564, filed Dec. 22, 2020, now U.S. Pat. No. 12,297,447, which is a Continuation of U.S. patent application Ser. No. 15/537,384, filed Jun. 16, 2017, now U.S. Pat. No. 10,876,134, which is a National Stage Entry of International Application No. PCT/US2015/066206, filed Dec. 16, 2015, which claims the benefit of U.S. Provisional Patent Application No. 62/092,501, filed Dec. 16, 2014, and U.S. Provisional Patent Application No. 62/146,793, filed Apr. 13, 2015. The foregoing are incorporated by reference in their entireties.

### SEQUENCE LISTING

The instant application contains a Sequence Listing, which has been submitted electronically in XML format and is hereby incorporated by reference in its entirety. Said XML copy, created on Dec. 18, 2025, is named 135767-0111_SL.xml and is 78,643 bytes in size.

### BACKGROUND

Lysosomal storage diseases are a class of metabolic disorders caused by mutations in proteins important for lysosomal function. There are many types of lysosomal storage diseases, and although each is relatively rare, their combined prevalence is estimated to be 1 in 8,000 live births (Schultz et al. (2011) Trends Neurosci., 34:401-410). Juvenile neuronal ceroid lipofuscinosis (JNCL or Juvenile Batten disease) is a fatal, neurodegenerative lysosomal storage disease appearing in about 1 in 100,000 live births that typically presents in children between the ages of 5-10 years. JNCL initiates as blindness and progresses to seizures, motor loss, and subsequent cognitive decline (Getty and Pearce (2011) Cell. Mol. Life Sci. 68:453-474).

Juvenile Batten Disease is caused by an autosomal recessive mutation in the (IN3 gene, most commonly due to a 1.02 kb deletion in exons 7 and 8 (Int. Batten Dis. Consortium (1995) Cell, 82:949-957), and although the CLN3 protein has been shown to reside in lysosomal membranes and other membrane compartments, its function remains elusive. CLN3 has been implicated in several cellular processes including, inter alia, endocytosis and intracellular protein trafficking, lysosomal homeostasis, autophagy, mitochondrial function, amino acid transport, oxidative stress, neuronal excitotoxicity, and cell cycle regulation.

Juvenile Batten Disease is characterized by the abnormal intracellular accumulation of lipid and protein (ceroid lipofuscin) in lysosomes, resulting in the development of insoluble inclusions. Although lysosomal inclusions form in all cell types in the body, neurons are the most sensitive and undergo progressive cell death (Getty and Pearce (2011) Cell. Mol. Life Sci. 68:453-474). The seriousness of this neuronal cell death is magnified by the fact that the central nervous system (CNS) is not capable of regeneration. Currently, there is no treatment for Juvenile Batten Disease, which is uniformly fatal and associated with a decreased life expectancy into the late teens or early twenties.

### SUMMARY

Compositions and methods for the treatment of Juvenile Neuronal Ceroid Lipofuscinosis (JNCL), also known as Juvenile Batten Disease and associated disorders and symptoms are provided herein. In certain embodiments the compositions comprise, or alternatively consist essentially of, or yet further consist of, adeno-associated viral (AAV) constructs, that in turn comprise, or alternatively consist essentially of, or yet further consist of, adeno-associated viral constructs, e.g., self-complementary or non-self-complementary adeno-associated viral (sc-AAV) constructs, that express a nucleic acid encoding human CLN3 (the human gene CLN3 (or a CLN3 cDNA)). In one illustrative, but non-limiting embodiment, an AAV-9 construct or a scAAV-9 construct is used. The AAV constructs comprise, in various embodiments, a variety of promoters and/or promoter/enhancers combinations can be used within the AAV construct to drive expression of the human-CLN3 gene or CLN3 cDNA or an equivalent of each thereof. Examples of promoters that can be used within the AAV construct include, but are not limited to, the β-actin promoter (e.g., the chicken β-actin promoter), the MeCP2 promoter, and the like. The AAV-hCLN3 constructs can be used to restore expression of wild-type CLN3 in the brain and other key tissues or organs which in turn can help treat JNCL and/or improve symptoms associated with JNCL. This gene therapy can be administered through a variety of routes including, inter alia, intravenous injection, intracranial injection, intrathecal injection, and the like.

Various embodiments contemplated herein may include, but need not be limited to, one or more of the following:

In one aspect, the disclosure provides a vector comprising an adeno-associated virus (AAV) genome or a derivative thereof and a promoter operably linked to a polynucleotide sequence encoding CLN3, e.g. a human CLN3, or an equivalent of each thereof.

In another aspect, the disclosure provides a vector comprising a self-complementary AAV9 genome or a derivative thereof and a promoter operably linked to a polynucleotide sequence encoding CLN3, wherein the promoter drives low CLN3 expression.

In another aspect, the disclosure provides a method for the treatment and/or prophylaxis of Juvenile neuronal ceroid lipofuscinosis (JNCL) in a mammal, the method comprising, or alternatively consisting essentially of, or alternatively consisting of, transforming cells of the mammal with a construct that expresses CLN3 where the CLN3 is expressed at an effective amount to treat and/or prevent JNCL

In some embodiments, the CLN3 is a human CLN3. In other embodiments, the CLN3 is a non-human CLN3.

In some embodiments, the AAV genome is from a naturally derived serotype or an isolate or a clade of AAV. In some embodiments, the AAV serotype is selected from the group of AAV1, AAV2, AAV4, AAV5, AAV6, AAV8, or AAV9. In preferred embodiments, the AAV serotype is AAV9. In other embodiments, the AAV serotype is AAV2.

In some embodiments, the vector is a derivative of AAV, e.g., a single-stranded AAV (ss-AAV) vector. In other embodiments, the derivative vector is a self-complementary AAV (sc-AAV) vector.

In some embodiments, the promoter is selected from a cytomegalovirus (CMV), a chicken β-actin (CBA), an Ubiquitin C (UBC), a β-glucuronidase (GUSB), a neuron-specific enolase (NSE), a Synapsin, a MeCP2 (methyl-CPG binding protein 2), a glial fibrillary acidic protein (GFAP), a β-actin 9 (e.g. chicken beta actin), or a CBh (hybrid CBA or a MVM intron with CBA promoter).

In some embodiments, the promoter is a neuron-, astrocyte-, or oligodendrocyte-specific or neuron-, astrocyte-, or oligodendrocyte-preferential promoter, for example, an NSE, a Synapsin, a MeCP2, an oligodendrocyte transcription factor 1 (Olig1), a chondroitin sulfate proteoglycan (Cspg4), a CNP (2′,3′-Cyclic-nucleotide 3′-phosphodiesterase), or a GFAP promoter.

In some embodiments, the vector further comprises, or alternatively consists essentially of, or yet further consists of a 5′UTR/intron selected from SV40 or CBA-MVM. In some embodiments, the vector further comprises, or alternatively consists essentially of or yet further consists of, a minimal SV40 intron. In other embodiments, the vector further comprises or alternatively consists essentially of, or yet further consists of a polyadenylation signal selected from a bovine growth hormone polyadenylation sequence, a SV40 late polyadenylation sequence, a SV40 early polyadenylation sequence, an AATAAA (SEQ ID NO: 3) polyadenylation signal, a CAATAAA (SEQ ID NO: 4) polyadenylation signal, an ATTAAA (SEQ ID NO: 5) polyadenylation signal, or a TANA (SEQ ID NO: 6) polyadenylation signal. In yet other embodiments, the vector further comprises or alternatively consists essentially of, or yet further consists of a posttranslational regulatory element, non-limiting examples of such include for example, a Woodchuck Post-transcriptional Regulatory Element (WPRE), a WPRE2 containing a minimal gamma element and a partial alpha-beta element, a WPRE3 and containing minimal gamma and alpha elements, or a hepatitis B virus posttranscriptional regulatory element (HPRE).

In some embodiments, the vector comprises one or more of the above elements and the vector is an AAV9 comprising a self-complementary genome.

In some embodiments, the polynucleotide encoding CLN3, e.g., human CLN3, or an equivalent thereof is operably linked to a MeCP2 promoter.

In some embodiments, the vector drives low CLN3 expression or an equivalent thereof.

In a further aspect, the vectors as described herein further comprise a gene or polypeptide that is a detectable label. Examples of such are exemplified in FIG. 1.

In some embodiments, the disclosure provides methods and uses of the vectors as describe herein, which methods comprise transforming cells of the central nervous system of the mammal with a vector as described herein. In other embodiments, the method comprises transforming cells contained within non-CNS tissue(s) with a vector as described herein.

In some embodiments, the method is performed on a mammal that in one aspect is a human and the CLN3 is a human CLN3 or an equivalent thereof.

In some embodiments, the human to be treated is homozygous for a CLN3 mutation.

In a further aspect, the CLN3 mutation is related to the development of Batten disease in the human bearing the homozygous mutation.

In some embodiments, the mammal to be treated is a human neonate, a human infant, or a human adolescent. In other embodiments, the mammal to be treated is a human adult.

In some embodiments, the mammal, such as a human patient to be treated, is asymptomatic for JNCL. In other embodiments, the mammal to be treated is symptomatic for JNCL, for example, the mammal presents with blindness, seizures, motor loss, cognitive decline, or any combination thereof.

The methods as disclosed herein comprise, or alternatively consist essentially of, or yet further consists of, administering to the mammal in need of such treatment and/or prophylaxis an effective amount of a vector, and/or an rAAV, and/or a pharmaceutical formulation containing the vector according to any one of the embodiments as described herein.

In some embodiments, the administration is via a route selected from one or more of intracerebral administration, intrathecal administration), intravenous, oral, aerosol administration, administration via inhalation (including intranasal and intratracheal delivery), intravenous, intramuscular, subcutaneous, intradermal, and rectal administration.

In some embodiments, the treatment regimen comprises a single administration, for example, a single systemic administration. In other embodiments, the treatment regimen comprises at least 2, at least 3, at least 4, at least 5, at least 6, at least 7, at least 8, at least 9, or at least 10 administrations. The dosing of the vector for each administration may be the same or different, as determined by the treating physician or professional.

In some embodiments, each administration comprises about 10¹⁰ up to about 10¹⁶ genome copies of CLN3 or an equivalent thereof per kg of body weight of the mammal to be treated. In other embodiments, each administration comprises about 10¹⁰ up to about 10¹⁶ genome copies of CLN3 or an equivalent thereof per subject.

In one aspect, the disclosure provides a host cell transduced with a vector according to any one of the embodiments as described herein.

In one aspect, the disclosure provides a method of transducing cells, the method comprising, or alternatively consisting essentially of, or yet further consisting of introducing into a host cell, a composition comprising an AAV vector according to any one of the embodiments as described herein

In another aspect, the disclosure provides a recombinant adeno-associated virus that comprises a nucleic acid construct that encodes human CLN3 or an equivalent thereof. In one embodiment, the adeno-associated virus comprises a vector according to any one of the embodiments as described herein.

In another aspect, the disclosure provides a recombinant adeno-associated virus according to any one of the above embodiments and a carrier, such as a pharmaceutically acceptable carrier or diluent. In some embodiments, the formulation is formulated for administration via a route selected from the group consisting of intracerebral administration, intrathecal administration, intravenous, oral, aerosol administration, administration via inhalation (including intranasal and intratracheal delivery), intravenous, intramuscular, subcutaneous, intradermal, and rectal administration. In other embodiments, the formulation is a sterile injectable.

In another aspect, the disclosure provides for the use of the vector of any one of the embodiments as described herein for the treatment and/or prophylaxis Juvenile Neuronal Ceroid Lipofuscinosis (JNCL).

### BRIEF DESCRIPTION OF THE FIGURES

FIG. 1 depicts exemplary AAV9 hCLN3 constructs. Each construct contains identical minimal SV40 intron (labeled “intron”), human CLN3 cDNA, bovine growth hormone polyadenylation signals (“PA”), and viral inverted terminal repeats to package self-complementary (sc) virus. The first construct contains the high expressing chicken β-actin promoter, while the second contains the minimal essential promoter from the mouse MeCP2 gene. Control constructs replace human CLN3 cDNA with a polynucleotide encoding green fluorescent protein (GFP).

FIGS. 2A-FIG. 2F, show that CLN3 replacement using scAAV9/MeCP2-hCLN3 improves motor behaviors in CLN3^{Δex7/8 mic}. One month-old CLN3^{Δex7/8} mice (8/group) received one i.v. injection of 2×10¹² viral genomes (vg) of scAAV9/MeCP2-hCLN3, scAAV9/β-actin-hCLN3, or GFP expressing viruses as controls, whereupon accelerating rotarod and pole descent tests were performed at 1 month (FIG. 2A), 2 months (FIG. 2B), 3 months (FIG. 2C), 4 months (FIG. 2D), and 5 months (FIG. 2E). *, p<0.05. FIG. 2F summarizes the data depicted in FIGS. 2A-2E showing improved motor behavior in CLN3^{Δex7/8} mice receiving scAAV9/MeCP2-hCLN3.

FIG. 3 illustrates dose-dependent hCLN3 expression in the brain following scAAV9 transduction. One month-old CLN3^{Δex7/8} mice (4/group) received one i.v. injection of 2×10¹² vg scAAV9/β-actin-hCLN3 or scAAV9/MeCP2-hCLN3, whereupon hCLN3 mRNA expression was quantitated 5 months later in the indicated brain regions (CB, cerebellum; TH, thalamus; HPC, hippocampus; STR, striatum; and VC, visual cortex) by qRT-PCR. Results are expressed as the fold-increase in hCLN3 expression in mice receiving scAAV9/β-actin-hCLN3 compared to scAAV9/MeCP2-hCLN3.

FIG. 4 illustrates dose-dependent GFP expression in the brain following scAAV9 transduction. One month-old CLN3^{Δex7/8} mice (3/group) received one i.v. injection of 2×10¹² vg scAAV9/β-actin-GFP or scAAV9/MeCP2-GFP, whereupon GFP expression was quantitated 5 months later in the indicated brain regions (CB, cerebellum; TH, thalamus; HPC, hippocampus; STR, striatum; VC, visual cortex; SIBF, somatosensory barrel field cortex) by Western blot with β-actin as a loading control.

FIGS. 5A-5B show biodistribution scAAV9/β-actin-GFP or scAAV9/MeCP2-GFP as visualized by confocal microscopy in brain regions and retina 5 months and 13 months post-transduction, respectively.

FIGS. 6A-6B show comparative biodistribution of scAAV9/β-actin-GFP or scAAV9/MeCP2-GFP in the brains of CLN3^{Δex7/8} mice 5 months after administration as depicted by number of GFP⁺ cells.

FIGS. 7A-7G show that CLN3 replacement reduces lysosomal inclusions. One month-old CLN3^{Δex7/8} mice (4/group) received one i.v. injection of 2×10¹² vg scAAV9/β-actin-hCLN3 or scAAV9/MeCP2-hCLN3, whereupon lysosomal inclusions were quantitated 5 months later in somatosensory barrel field cortex (S1BF) (FIG. 7A), striatum (STR) (FIG. 7B), visual cortex (VC) (FIG. 7C), thalamus (TH) (FIG. 7D), hippocampus (HP) (FIG. 7E), cerebellum (CB) (FIG. 7F) by quantitative confocal microscopy. *, p<0.05. A summary of the comparison of vehicle and MeCP2-hCLN3 in SIBF, VC, and TH is shown in FIG. 7G).

FIGS. 8A-8C shows the effect of systemic administration of scAAV9 on transduction of brain tissue. FIG. 8A shows that systemic administration of scAAV9 transduces neurons in the CLN3^{Δex7/8} brain. One month-old CLN3^{Δex7/8} mice (4/group) received one injection of 2×10¹² vg scAAV9/MeCP2-GFP i.v., whereupon brain tissues were collected 5 months later and stained for NeuN, GFP, and nuclei (DAPI) in the indicated brain regions for confocal microscopy. Insets depict regions indicated at higher magnification. FIG. 8B shows that systemic administration of scAAV9 transduces astrocytes in the CLN3^{Δex7/8} brain. FIG. 8C shows that systemic administration of scAAV9 does not transduce microglia in the CLN3^{Δex7/8} brain.

FIG. 9 shows that GFAP reactive astrocytes, a hallmark of human Juvenile Batten Disease and also observed in the CLN3^{Δex7/8} brain, are significantly reduced in mice following the systemic administration of scAAV9/MeCP2-hCLN3. One month-old CLN3^{Δex7/8} mice (4/group) received one i.v. injection of scAAV9/MeCP2-hCLN3, whereupon reactive astrocytes (GFAP) were quantitated 5 months later in the somatosensory barrel field cortex by quantitative confocal microscopy. ***, p<0.001.

FIG. 10 show that CLN3 replacement using scAAV9/MeCP2-hCLN3 improves motor behaviors in CLN3^{Δex7/8 mic}. One month-old CLN3^{Δex7/8} mice (8/group) received one i.v. injection of 2×10¹² vg of scAAV9/MeCP2-hCLN3, scAAV9/β-actin-hCLN3, or GFP expressing viruses as controls, whereupon adhesive tape removal tests were performed at 11 months post-transduction showing reduced time to touch (left panel) and reduced time to remove (right panel) in CLN3^{Δex7/8} mice receiving scAAV9/MeCP2-hCLN3.

FIG. 11 illustrates effect of system AAV9 transgene delivery to CLN3^{Δex7/8} mice of AAV9/β-actin-hCLN3, scAAV9/MeCP2-hCLN3, AAV9/β-actin-GFP scAAV9/MeCP2-GFP. Results are expressed as weight in grams as determined weekly.

FIG. 12 demonstrates normal serum chemistry profiles for CLN3^{Δex7/8} mice administered AAV9/β-actin-hCLN3, scAAV9/MeCP2-hCLN3, AAV9/β-actin-GFP, or scAAV9/MeCP2-GFP 1-month post-infection.

FIG. 13 demonstrates normal serum chemistry profiles for CLN3^{Δex7/8} mice administered AAV9/β-actin-hCLN3, scAAV9/MeCP2-hCLN3, AAV9/β-actin-GFP, or scAAV9/MeCP2-GFP 10-months post-infection.

FIG. 14 demonstrates that CLN3^{Δex7/8} mice administered with AAV9/β-actin-hCLN3, scAAV9/MeCP2-hCLN3, AAV9/β-actin-GFP, or scAAV9/MeCP2-GFP show no evidence of systemic inflammatory changes.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

### I. Definitions

As used herein the following terms have the following meanings.

The term “about” when used before a numerical designation, e.g., temperature, time, amount, concentration, and such other, including a range, indicates approximations which may vary by (+) or (−) 10%, 5% or 1%.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, the term “vector” refers to a polynucleotide construct, typically a plasmid or a virus, used to transmit genetic material to a host cell. Vectors can be, for example, viruses, plasmids, cosmids, or phage. A vector as used herein can be composed of either DNA or RNA. In some embodiments, a vector is composed of DNA. An “expression vector” is a vector that is capable of directing the expression of a protein encoded by one or more genes carried by the vector when it is present in the appropriate environment. Vectors are preferably capable of autonomous replication. Typically, an expression vector comprises a transcription promoter, a gene, and a transcription terminator. Gene expression is usually placed under the control of a promoter, and a gene is said to be “operably linked to” the promoter.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The terms “nucleic acid” or “polynucleotide” are used interchangeably herein. In some embodiments, the nucleic acid comprises at least two nucleotides covalently linked together. In some embodiments, the nucleic acid of the present invention is single-stranded. In some embodiments, the nucleic acid is double stranded. In some embodiments, the nucleic acid is triple-stranded. In some embodiments, the nucleic acid comprises phosphodiester bonds. In some embodiments, the nucleic acid comprises a single-stranded or double-stranded deoxyribonucleic acid (DNA) or a single-stranded or double-stranded ribonucleic acid (RNA). In some embodiments, the nucleic acid comprises a nucleic acid analog. In some embodiments, the nucleic acid analog has a backbone, comprising a bond other than and/or in addition to a phosphodiester bond, such as, by non-limiting example, phosphoramide, phosphorothioate, phosphorodithioate or O-methylphophoroamidite linkage. In some embodiments, the nucleic acid analog is selected from a nucleic acid analog with a backbone selected from a positive backbone; a non-ionic backbone and a non-ribose backbone. In some embodiments, the nucleic acid contains one or more carbocyclic sugars. In some embodiments, the nucleic acid comprises modifications of its ribose-phosphate backbone. In some embodiments, these modifications are performed to facilitate the addition of additional moieties such as labels. In some embodiments, these modifications are performed to increase the stability and half-life of such molecules in physiological environments.

The term “regulatory element” and “expression control element” are used interchangeably and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Illustrative, but non-limiting regulatory elements in prokaryotes include promoters, operator sequences ribosome binding sites, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription. In certain embodiments, the enhancer acts to increase transcription regardless of the distance or orientation of the enhancer relative to the start site of transcription.

As used herein, the term “transfection” is synonymous with “transducing” and refers to the introduction of a nucleic acid, such as an exogenous nucleic acid, into a host cell. In certain embodiments the introduction is by contacting the cell with a recombinant AAV virus or vector comprising the nucleic acid (e.g., an expression cassette) that is to be introduced. An AAV genome is a genome that is in whole or in part derived from an adeno-associated virus that infects humans and other primates. The AAV genome is comprised of single-stranded DNA (ssDNA), either positive-or negative-sensed. The genome comprises inverted terminal repeats (ITRs) at both ends of the DNA and two open reading frames. A self-complementary AAV (scAAV) is a viral vector created from wild-type AAV. The self-complementary AAV is an example of a AAV derivative and is an engineered viral vector derived from AAV that has been designed to form an intra-molecular double-stranded DNA template.

As used herein, the term “transgene” refers to any nucleotide or DNA sequence that is transfected into a target cell. The construct comprising the transgene may exist as a stable episome, or may be integrated into one or more chromosomes of a target cell. In some embodiments, the transgene comprises a polynucleotide that encodes a protein of interest (e.g. CLN3). The polynucleotide encoding the desired protein is generally operatively linked to other sequences that are useful for obtaining the desired expression of the gene of interest, such as promoters, enhancers, transcriptional regulatory sequences, and the like.

As used herein, the ceroid-lipofuscinosis, neuronal 3 gene or (“CLN3 gene”) intends a nucleic acid that encodes a protein that is involved in lysosomal function. Mutations in this gene cuase neurodegenerative diseases. The nucleic acid encoding the human protein and the human protein are known in the art and provided at GenBank Accession No. NM_001042432 (last accessed on Dec. 15, 2015), and examples of non-human CLN3 genes are the rat and mouse homologs, that are available at the website genenames.org/cgi-bin/gene_symbol_report?hgnc_id=HGNC: 2074, last accessed on Dec. 15, 2015.

The terms “treatment, treat, treating, etc.” as used herein, include but are not limited to, alleviating a symptom of a disease or condition (e.g., Juvenile neuronal ceroid lipofuscinosis (JNCL or Juvenile Batten disease)) and/or reducing, suppressing, inhibiting, lessening, ameliorating or affecting the progression, severity, and/or scope of the disease or condition. “Treatments” refer to one or both of therapeutic treatment and prophylactic or preventative measures. Subjects in need of treatment include those already affected by a disease or disorder or undesired physiological condition as well as those in which the disease or disorder or undesired physiological condition is to be prevented.

The term “effective amount,” as used herein, refers to an amount that is capable of treating or ameliorating the disease or condition or otherwise capable of producing an intended therapeutic effect. In certain embodiments, an effective amount is an amount sufficient to reduce, prevent, or delay the onset of one or more symptoms of JNCL. Such symptoms may include, but are not limited to vision problems or blindness, seizures, personality and/or behavior changes, slow learning, clumsiness, stumbling, mental impairment, and loss of motor skills.

The terms “patient,” “subject,” or “mammalian subject” are used interchangeably herein and include any mammal in need of the treatment or prophylactic methods described herein (e.g., methods for the treatment or prophylaxis of JNCL). Such mammals include, particularly humans (e.g., fetal humans, human infants, human teens, human adults, etc.). Other mammals in need of such treatment or prophylaxis can include non-human mammals such as dogs, cats, or other domesticated animals, horses, livestock, laboratory animals (e.g., lagomorphs, non-human primates, etc.), and the like. The subject may be male or female. In certain embodiments the subject is at risk, but asymptomatic for JNCL (e.g., a child that has a genetic diagnosis of JNCL before clinical signs are apparent (this rarely occurs), or a child that has a genetic diagnosis of JNCL at an age before clinical symptoms develop (i.e. birth to around 4-5 years), e.g., where genetic testing is triggered by an older sibling with a confirmed diagnosis of JNCL). In certain embodiments, the subject expresses symptoms of JNCL.

The term “administering” or “administration” of vector to a subject includes any route of introducing or delivering to a subject the vector to perform its intended function. Administration can be carried out by any suitable route, including orally, intranasally, parenterally (intravenously, intramuscularly, intraperitoneally, or subcutaneously), intracranially, or topically. Administration includes self-administration and the administration by another.

The terms “polynucleotide,” “nucleic acid” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein +SPupdate+PIR. Details of these programs can be found at the following Internet address: ncbi.nlm.nih.gov/cgi-bin/BLAST.

An equivalent or biological equivalent nucleic acid, polynucleotide or oligonucleotide or peptide is one having at least 70% sequence identity, or alternatively at least 80% sequence identity, or alternatively at least 85% sequence identity, or alternatively at least 90% sequence identity, or alternatively at least 92% sequence identity, or alternatively at least 95% sequence identity, or alternatively at least 97% sequence identity, or alternatively at least 98% sequence identity to the reference nucleic acid, polynucleotide, oligonucleotide or peptide. An alternative equivalent polypepeptide is a polypeptide encoded by a nucleic acid that hybridizes under conditions of high stringency to a reference polynucleotide or its complement. An alternative equivalent polynucleotide is one that hybridizes under conditions of high stringency to a reference polynucleotide or its complement.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency”. In general, a low stringency hybridization reaction is carried out at about 40° C. in 10×SSC or a solution of equivalent ionic strength/temperature. A moderate stringency hybridization is typically performed at about 50° C. in 6×SSC, and a high stringency hybridization reaction is generally performed at about 60° C. in 1×SSC. Additional examples of stringent hybridization conditions include: low stringency of incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Hybridization reactions can also be performed under “physiological conditions” which is well known to one of skill in the art. A non-limiting example of a physiological condition is the temperature, ionic strength, pH and concentration of Mg²⁺ normally found in a cell.

As used herein, the term “low expression” intends less than that expressed using a “high expression” promoter such as the β-actin or CMV promoter that are known in the art to drive gene expression at higher than the native promoter. A non-limiting of a low expression promoter is the MeCP2 promoter.

### Modes for Carrying Out the Disclosure

Juvenile Neuronal Ceroid Lipofuscinosis (JNCL) or Juvenile Batten Disease is a lysosomal storage disorder caused by an autosomal recessive mutation in CLN3. JNCL presents between 5-10 years of age, with progressive vision loss, seizures, cognitive and motor decline, and death occurring by the late teens to early 20s. There is no cure for JNCL, which underscores the significance of identifying novel therapeutics to improve lifespan and quality-of-life for children suffering from this deadly disease.

Current therapeutic approaches for JNCL target pathological sequelae. However, to provide long-lasting protection, alternative strategies are required. One such approach is gene therapy. Gene therapy has shown promise in correcting some forms of Batten Disease that involve mutations in soluble enzymes, in part, due to cross-correction of neighboring non-transduced cells. However, since CLN3 is a transmembrane protein, gene therapy approaches have been considered less practical for JNCL. Despite this, recent evidence has suggested that gene therapy for JNCL may be feasible, where the intracranial delivery of an adenoassociated virus (AAV) vector harboring human CLN3 was capable of reducing lysosomal inclusions, although no changes in behavioral deficits were reported.

As illustrated herein in the Examples, AAV9/hCLN3 constructs have been engineered using the β-actin or MeCP2 promoters to drive high versus low expression of hCLN3, respectively. One (1) month-old CLN3^{Δex7/8} mice were injected with 2×10¹² viral genomes (vg) of AAV9/β-actin-hCLN3 or AAV9/MeCP2-hCLN3 intravenously, with viruses driving green fluorescence protein (GFP) expression (AAV9/β-actin-GFP and AAV9/MeCP2-GFP) as controls.

Importantly, a promoter-dosage effect for hCLN3 was confirmed in several brain regions where neurons are destined to die in JNCL, including the hippocampus (HPC), striatum (STR), thalamus (TH), visual cortex (VC), and cerebellum (CB), where hCLN3 expression was elevated 3- to 8-fold in CLN3^{Δex7/8} mice receiving high expressing AAV9/β-actin-hCLN3 vs. AAV9/MeCP2-hCLN3. Animals receiving AAV9/GFP constructs revealed that the virus mainly transduced NeuN neurons, with a few GFAP astrocytes also observed, whereas GFP expression was not detected in microglia. The extent of viral transduction in the brain was widespread, as evident by GFP expression in the somatosensory barrel field cortex (SIBF), VC, STR, TH, HPC, SCN, and CB.

Importantly, only the AAV9 construct driving low hCLN3 expression (AAV9/MeCP2-hCLN3) was capable of restoring motor deficits of CLN3^{Δex7/8} mice (accelerating rotarod and pole climbing) to performance levels typical of WT animals, demonstrating that high CLN3 expression in the brain was not advantageous for improved motor coordination. Similar benefits were observed with lysosomal storage material, which was significantly reduced in the SIBF, VC, and TH of animals receiving AAV9/MeCP2-hCLN3. The beneficial effects of AAV9/MeCP2-hCLN3 on motor behavior in CLN3^{Δex7/8} mice were not observed with GFP control constructs, confirming specificity of action for hCLN3. Based on these observations and without being bound by theory, it is believed that AAV/hCLN3 (e.g., AAV9/hCLN3)-mediated gene therapy will target sufficient numbers of CNS cells to significantly improvement JNCL outcome. In this regard, it is noted that heterozygous carriers of the CLN3 mutation (i.e. 1/2 gene dosage) do not have any evidence of disease or pathology indicating that these “reduced levels of CLN3 expression” are sufficient to produce healthy cells. The scAAV9/MeCP2-GFP virus described herein has been used to identify the frequency of transduced cells in the CNS. Albeit at a modest frequency, the method used to identify cells transduced with viral constructs (i.e. immunofluorescence staining with GFP antibody) has limited sensitivity suggesting that it is possible that the number of cells in the brain that were transduced may be underestimated. Nevertheless, the dramatic improvements in disease readouts described herein indicate that the AAV gene therapy approach(es) described herein are effective.

Accordingly in various embodiments, AAV/hCLN3 constructs, rAAV that express hCLN3, and methods of treatment and/or prophylaxis utilizing AAV/hCLN3 constructs are provided.

### AAV Vectors and Compositions

In certain embodiments the polynucleotide encoding a CLN3 protein is delivered to cells within or comprising one or more tissues of the central nervous system (CNS) by means of a viral vector, of which many are known and available in the art. Additionally, in various embodiments CLN3 will also be delivered to systemic tissues, which is important because children with Juvenile Batten Disease also have systemic disease symptoms (i.e. cardiac defects). In this regard it is noted that CLN3 is ubiquitously expressed in all cell types in the body. The viral vector(s) are desirably non-toxic, non-immunogenic, easy to produce, and efficient in protecting and delivering DNA into the target cells. In one illustrative, but non-limiting embodiment, the viral vector is an adeno-associated virus vector or a derivative thereof and therapeutic compositions comprising adeno-associated viral vector or a derivative thereof comprising the nucleic acid sequence encoding CLN3 polypeptide is under the control of a suitable promoter.

More than 30 naturally occurring serotypes of AAV are available and are useful in the constructs and methods as described herein. Many natural variants in the AAV capsid exist, allowing identification and use of an AAV with properties specifically suited for neural cells as well as other cell types. AAV viruses can be engineered by conventional molecular biology techniques, making it possible to optimize these particles for cell specific delivery of the desired nucleic acid sequences, for minimizing immunogenicity, for tuning stability and particle lifetime, for efficient degradation, for accurate delivery to the nucleus, etc.

The use of AAVs is a common mode of exogenous delivery of DNA as it is relatively non-toxic, provides efficient gene transfer, and can be easily optimized for specific purposes. Among the serotypes of AAVs isolated from human or non-human primates (NHP) and well characterized, human serotype 2 is the first AAV that was developed as a gene transfer vector. This serotype has been widely used for efficient gene transfer experiments in different target tissues and animal models. Other AAV serotypes include useful in the vectors and methods of this disclosure include, but are not limited to, AAV1, AAV3, AAV4, AAV5, AAV6, AAV6.2, AAV7, AAV8, AAV9, rh. 10, rh.39, rh.43, CSP3, and the like (see, e.g., WO 2005/033321 and U.S. Pat. No. 7,198,951) for a discussion of various AAV serotypes). In certain embodiments the serotype is selected to optimize the desired mode of delivery. For example, AAV1, AAV5, and AAV9 provide the good vector spread and high efficiency of transduction. AAV1 and AAV9 provide almost exclusively neuronal tropism, while AAV5 provides a mix of neurons and glia, and AAV4 targets mostly astrocytes (see, e.g., Davidson et al. (2000) Proc. Natl. Acad. Sci. USA, 97:3428-3432); Burger et al. (2004) Mol. Ther. 10:302-317; Cearley and Wolfe (2006) Mol. Ther. 13:528-537). AAV1 and AAV6 have superior retrograde axonal transport capabilities following peripheral injection (Hollis et al. (2008) Mol. Ther. 16:296-301), while AAV9 undergoes efficient axonal transport within the brain (Cearley and Wolfe, (2006) supra.). AAV6, AAV8, and AAV9 have demonstrated efficient delivery to the spinal cord and dorsal root ganglia following intrathecal administration, targeting different subsets of cells depending on the specific serotype (Storek et al. (2008) Proc. Natl. Acad. Sci. USA, 105:1055-1060; Towne et al. (2009) Mol. Pain, 5:52; Snyder et al. (2011) Hum. Gene Ther., 22:1129-1135). Intracerebral ventricular injection of AAV4 efficiently transduces ependymal cells (Liu et al. (2005) Gene Ther. 12:1503-1508). As demonstrated herein AAV9 can cross the blood-brain barrier (BBB) following intravenous administration to transduce neurons and glia within the brain. In certain embodiments the rh. 10 serotype is expressly excluded. Thus, it is within the scope of this disclosure that dosages of different AAV serotypes can be administered to the patient concurrently and/or sequentially, to provide targeted delivery to personalize the therapy based on the symptoms and progression of the disease.

Desirable AAV fragments for assembly into vectors include the cap proteins known in the art, including the vp1 (e.g., SEQ ID NOs: 12 and 13), vp2 (e.g., SEQ ID NOs: 14 and 15), vp3 (e.g., SEQ ID NOs: 16 and 17) and hypervariable regions, the rep proteins, including rep 78 (e.g., SEQ ID NO: 18), rep 68 (e.g., SEQ ID NO: 19), rep 52 (e.g., SEQ ID NO: 20), and rep 40 (e.g., SEQ ID NO: 21), and the sequences encoding these proteins and equivalents thereof. These fragments may be readily utilized in a variety of vector systems and host cells. Such fragments maybe used, alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique. Using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a pseudotyped AAV, a chimeric AAA″ capsid, a recombinant AAV capsid, or a “humanized” AAV capsid. Pseudotyped vectors, in which the capsid of one AAV is replaced with a heterologous capsid protein, also useful. (see, e.g. Asokan A. (2010) Discov. Med. 9 (48): 399-403).

In one illustrative, but non-limiting embodiment, the vectors useful in compositions and methods described herein contain, at a minimum, sequences encoding a selected AAV serotype capsid, e.g., an AAV9 capsid (e.g., SEQ ID NO: 13), or a fragment or equivalent thereof. In another embodiment, useful vectors contain, at a minimum, sequences encoding a selected AAV serotype rep protein, e.g., AAV9 rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAV9 origin. (see, e.g. Balakrishnan et al. (2014) Curr. Gene. Therap. 14:1-15; U.S. Pat. Nos. 8,962,330; and 7,198,951).

Alternatively, vectors may be used in which the rep sequences are from an AAV serotype that differs from that which is providing the cap sequences. In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199. In some embodiments, an AAV1 rep protein is fused in frame to cap sequences of the AAV serotype selected from the group consisting of AAV2, AAV3, AAV4, AAV5, AAV6, AAV6.2, AAV7, AAV8, AAV9, rh. 10, rh.39, rh.43, and CSP3. In some embodiments, an AAV2 rep protein is fused in frame to cap sequences of the AAV serotype selected from the group consisting of AAV1, AAV3, AAV4, AAV5, AAV6, AAV6.2, AAV7, AAV8, AAV9, rh. 10, rh.39, rh.43, and CSP3. In some embodiments, an AAV3 rep protein is fused in frame to cap sequences of the AAV serotype selected from the group consisting of AAV1, AAV2, AAV4, AAV5, AAV6, AAV6.2, AAV7, AAV8, AAV9, rh. 10, rh.39, rh.43, and CSP3. In some embodiments, an AAV4 rep protein is fused in frame to cap sequences of the AAV serotype selected from the group consisting of AAV1, AAV2, AAV3, AAV5, AAV6, AAV6.2, AAV7, AAV8, AAV9, rh. 10, rh.39, rh.43, and CSP3. In some embodiments, an AAV5 rep protein is fused in frame to cap sequences of the AAV serotype selected from the group consisting of AAV1, AAV2, AAV3, AAV4, AAV6, AAV6.2, AAV7, AAV8, AAV9, rh. 10, rh.39, rh.43, and CSP3. In some embodiments, an AAV6 rep protein is fused in frame to cap sequences of the AAV serotype selected from the group consisting of AAV1, AAV2, AAV3, AAV4, AAV5, AAV6.2, AAV7, AAV8, AAV9, rh. 10, rh.39, rh.43, and CSP3. In some embodiments, an AAV6.2 rep protein is fused in frame to cap sequences of the AAV serotype selected from the group consisting of AAV1, AAV2, AAV3, AAV4, AAV5, AAV6, AAV7, AAV8, AAV9, rh. 10, rh.39, rh.43, and CSP3. In some embodiments, an AAV7 rep protein is fused in frame to cap sequences of the AAV serotype selected from the group consisting of AAV1, AAV2, AAV3, AAV4, AAV5, AAV6.2, AAV6.2, AAV8, AAV9, rh. 10, rh.39, rh.43, and CSP3. In some embodiments, an AAV8 rep protein is fused in frame to cap sequences of the AAV serotype selected from the group consisting of AAV1, AAV2, AAV3, AAV4, AAV5, AAV6.2, AAV6.2, AAV7, AAV9, rh. 10, rh.39, rh.43, and CSP3. In some embodiments, an AAV9 rep protein is fused in frame to cap sequences of the AAV serotype selected from the group consisting of AAV1, AAV2, AAV3, AAV4, AAV5, AAV6.2, AAV6.2, AAV7, AAV8, rh. 10, rh.39, rh.43, and CSP3. In some embodiments, an AAVrh. 10 rep protein is fused in frame to cap sequences of the AAV serotype selected from the group consisting of AAV1, AAV2, AAV3, AAV4, AAV5, AAV6.2, AAV6.2, AAV7, AAV8, AAV9, rh.39, rh.43, and CSP3. In some embodiments, an AAVrh.39 rep protein is fused in frame to cap sequences of the AAV serotype selected from the group consisting of AAV1, AAV2, AAV3, AAV4, AAV5, AAV6.2, AAV6.2, AAV7, AAV8, AAV9, rh. 10, rh.43, and CSP3. In some embodiments, an AAVrh.43 rep protein is fused in frame to cap sequences of the AAV serotype selected from the group consisting of AAV1, AAV2, AAV3, AAV4, AAV5, AAV6.2, AAV6.2, AAV7, AAV8, AAV9, rh. 10, rh.39, and CSP3. In some embodiments, an AAVCSP3 rep protein is fused in frame to cap sequences of the AAV serotype selected from the group consisting of AAV1, AAV2, AAV3, AAV4, AAV5, AAV6.2, AAV6.2, AAV7, AAV8, AAV9, rh. 10, rh.39, and rh.43.

Typically the vectors are composed of, at a minimum, a transgene and its necessary regulatory sequences, and 5′ and 3′ AAV inverted terminal repeats (ITRs). Non-limiting examples of ITRs include nucleotides 662 to 767 and nucleotides 3278 to 3418 of SEQ ID NO: 2 and equivalents of each thereof. The recombinant AAV vector is packaged into a capsid protein and delivered to a selected target cell. Thus, in one aspect, the vector contained within the capsid and/or target cell is an aspect of this disclosure. As contemplated herein, the transgene is one that encodes a CLN3 gene product (e.g., a CLN3 polypeptide (see, e.g., International Batten Disease Consortium (1995) Cell, 82:949-957, and GenBank Accession No: AAC05337 (SEQ ID NO:1), SEQ ID NO:7, SEQ ID NO:8, SEQ ID NO: 9, SEQ ID NO: 10, nucleotides 1683 to 2999 of SEQ ID NO: 2, and equivalents of each thereof. The nucleic acid coding sequence of the CLN3 gene product (e.g., SEQ ID NO: 11) and equivalents thereof are operatively linked to regulatory components in a manner that permits transgene transcription, translation, and expression in a cell of a target tissue.

The AAV sequences of the vector typically comprise the cis-acting 5′ and 3′ inverted terminal repeat sequences (see, e.g., Carter (1990) In Handbook of Parvoviruses, ed., P. Tijsser, CRC Press, pp. 155-168). The ITR sequences are typically about 145 bp in length. In certain embodiments substantially the entire sequences encoding the ITRs are used in the vector although some degree of modification of these sequences is permissible. The ability to modify these ITR sequences is within the skill of the art (see, e.g., Sambrook et al. (1989) Molecular Cloning. A Laboratory Manual, 2d ed., Cold Spring Harbor Laboratory, New York; and Fisher et al. (1996) J. Virol., 70:520-532, and the like). An example of such a molecule is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types. ***

In addition to the major elements identified above for the recombinant AAV vector, in certain embodiments, the vector can also typically include conventional control elements that are operably linked to the transgene in a manner that permits its transcription, translation and expression in a cell transfected with the plasmid vector or infected with the AAV virus. Expression control sequences include, for example, appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

For nucleic acid encoding proteins, a polyadenylation sequence generally is inserted following the transgene sequences and before the 3′ AAV ITR sequence. Suitable polyadenylation sequences include, but are not limited to bovine growth hormone polyadenylation sequence (e.g., nucelotides 3052 to 3198 of SEQ ID NO: 2, SV40 late polyadenylation sequence, SV40 early polyadenylation sequence, AATAAA (SEQ ID NO: 3) polyadenylation signal, CAATAAA (SEQ ID NO: 4) polyadenylation signal, ATTAAA (SEQ ID NO: 5) polyadenylation signal, TANA (SEQ ID NO: 6) polyadenylation signal.

In certain embodiments an AAV vector also contains an intron, desirably located between the promoter/enhancer sequence and the transgene. One illustrative, but non-limiting possible intron sequence is derived from SV-40, and is referred to as the SV-40 T intron sequence. Other intron sequences are known to those of skill in the art and include, inter alia, CBA, and CBA-MVM. (see, e.g., Gray et al. (2011) Hum. Gene Ther. 22 (9): 1143-1153 and references cited within).

Another element that can optionally be present is a post-translational regulatory element. Illustrative, post-translational regulatory elements include, but are not limited to Woodchuck Post-transcriptional Regulatory Element (WPRE), WPRE2 containing a minimal gamma element and a partial alpha-beta element, WPRE3 and containing minimal gamma and alpha elements (see, e.g., Choi et al. (2014) Molecular Brain 7:17-26). Non-limiting examples are provided in the attached sequence listing, and incorporated herein by reference.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors may optionally include 5′ leader or signal sequences. Examples of such signal peptides include the albumin, the β-glucuronidase, the alkaline protease or the fibronectin secretory signal peptides. In one aspect, the enhancer is a Woodchuck post-regulatory element (“WPRE”) (see, e.g., Zufferey, R. et al. (1999) J. Virol. 73 (4): 2886-2992). Non-limiting examples of WPREs include SEQ ID NO: 37 or SEQ ID NO: 38. The enhancer element can be downstream of the promoter and CLN3 gene. However, the enhancer can be in any location. Non-limiting examples are provided in the attached sequence listing, and incorporated herein by reference.

Examples of suitable promoters include, but are not limited to the retroviral Rous sarcoma virus (RSV) LTR promoter (optionally with the RSV enhancer) (e.g., SEQ ID No: 28 and equivalents thereof), the cytomegalovirus (CMV) promoter (optionally with the CMV enhancer) (see, e.g., Boshart et al (1985) Cell, 41:521-530 and e.g., SEQ ID No: 27 and equivalents thereof), the SV40 promoter (e.g., SEQ ID No: 29), the dihydrofolate reductase promoter, the β-actin promoter (e.g., chicken β-actin promoter) (e.g., SEQ ID No: 22, SEQ ID No: 23, and equivalents thereof),, the phosphoglycerol kinase (PGK) promoter (e.g., SEQ ID No: 30 and equivalents thereof), the EF1a promoter (e.g., SEQ ID No: 31 and equivalents thereof), the CBA promoter (e.g., SEQ ID No: 24 and equivalents thereof), UBC promoter (e.g., SEQ ID No: 34 and equivalents thereof), GUSB promoter, NSE promoter (e.g., SEQ ID No: 33 and equivalents thereof), Synapsin promoter (e.g., SEQ ID No: 32 and equivalents thereof), MeCP2 (methyl-CPG binding protein 2) promoter (e.g., SEQ ID No: 22, SEQ ID No: 23, and equivalents thereof), GFAP, CBh promoter and the like. It is contemplated that different promoters may be used to augment the expression level of the transgene (e.g., CLN3). For example, a chicken β-actin promoter (CBA) may result in strong, ubiquitous expression of the transgene, while a MeCP2 promoter may result in lower expression of the transgene as compared to another promoter. In some embodiments, the promoter may control transgene expression in a tissue-dependent manner. For example, promoter activity may be higher in neurons as compared to glia. In some embodiments, the promoter may control transgene expression in a developmental stage-dependent manner. For example, promoter-driven expression may rise postnatally and be essential throughout adult life.

In certain embodiments the native promoter, or fragment thereof, for the transgene can be used. The native promoter may be preferred when it is desired that expression of the transgene should mimic the native expression. In certain embodiments, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: neuronal such as neuron-specific enolase (NSE) promoter (Anderson et al. (1993) Cell. Mol. Neurobiol., 13:503-515) neurofilament light-chain gene promoter (Piccioli et al. (1991) Proc. Natl. Acad. Sci. USA, 88:5611-5615 and e.g., GenBank Accession No: L04147.1 (SEQ ID No:25)), and the neuron-specific vgf gene promoter (Piccioli et al. (1995) Neuron, 15:373-384 and GenBank Accession No: Y09938.I (SEQ ID No:26)). In some embodiments, the tissue-specific promoter is a promoter of a gene selected from: neuronal nuclei (NeuN), glial fibrillary acidic protein (GFAP) (Brenner et al. (1994) J. Neurosci. 14 (3): 1030-1037), adenomatous polyposis coli (APC), and ionized calcium-binding adapter molecule 1 (Iba-1). In some embodiments, the promoter is a chicken Beta-actin promoter (e.g., SEQ ID No: 24 or equivalents thereof) or an MeCP2 promoter (e.g., nucleotides 775 to 1511 of SEQ ID No: 2, SEQ ID No: 22, SEQ ID No: 23, or equivalents thereof).

In some embodiments, one or more binding sites for one or more of miRNAs are incorporated in a transgene of an AAV vector, to inhibit the expression of the transgene in one or more tissues of a subject harboring the transgenes, e.g., non-CNS tissues. The skilled artisan will appreciate that binding sites may be selected to control the expression of a transgene in a tissue specific manner. For example, expression of a transgene in the liver may be inhibited by incorporating a binding site for miR-122 such that mRNA expressed from the transgene binds to and is inhibited by miR-122 in the liver. Expression of a transgene in the heart may be inhibited by incorporating a binding site for miR-133a or miR-1, such that mRNA expressed from the transgene binds to and is inhibited by miR-133a or miR-1 in the heart. The miRNA target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. In certain embodiments the target site sequence may comprise a total of 5-100, 10-60, or more nucleotides. In certain embodiments the target site sequence may comprise at least 5 nucleotides of the sequence of a target gene binding site.

### The CLN3 Transgene

Ceroid-lipofuscinosis neuronal 3 (CLN3) is a hydrophobic, transmembrane protein found in the cellular membranes of multiple cellular structures, including lysosomal membranes, but molecular function of CLN3 is unknown. CLN3 has been implicated in several cellular processes, including endocytosis and intracellular protein trafficking, lysosomal homeostasis, autophagy, mitochondrial function, amino acid transport, oxidative stress, neuronal excitotoxicity, and cell cycle regulation. CLN3 is known to be important for the normal function of lysosomes-compartments within the cell that normally break down toxic substances and recycle any reusable components.

More than 60 mutations in the CLN3 gene have been identified in people with Batten disease. The most common mutation for Juvenile Batten Disease is an approximately 1 kb nucleotide deletion in the CLN3 gene. It is estimated that more than 90% of individuals with Juvenile Batten disease have such mutation. To study the disease, several animal models have been derived, including a CLN3 knockout (KO) mice and a CLN3^{Δex7/8} mouse (recapitulates the primary mutation observed in children).

As noted above, the constructs contemplated herein include a polynucleotide sequence that encodes human CLN3 (see, e.g., International Batten Disease Consortium (1995) Cell, 82:949-957, and GenBank Accession No: AAC05337 (SEQ ID NO:1)) and equivalents thereof. In some embodiments, the polynucleotide sequence is a CLN3 cDNA. It will be recognized, however, that the nucleic acid sequence encoding CLN3 may be altered, for example to facilitate construction of an expression cassette, to enhance expression (e.g., via coding optimization), and the like. In certain embodiments the encoded CLN3 peptide may be modified, e.g., to improve membrane insertion, to improve activity and/or stability, to reduce immunogenicity, and the like. In certain embodiments a non-human CLN3 may be utilized, particular for application of the AAV to a non-human subject for treatment or research purposes. It is noted that CLN3 sequences are known for a number of mammals including, but not limited to Canus lupus (e.g., GenBank Accession No: AAB05546.1 (SEQ ID NO: 7)), Mus musculus (e.g., GenBank Accession No: AAB69983 (SEQ ID NO: 8)), Ovis aries (e.g., GenBank Accession No: XP_011959725 (SEQ ID NO: 9)), Felis catus (e.g., GenBank Accession No: BAM42890 (SEQ ID NO: 10)), and the like.

In some embodiments, a suitable CLN3 nucleic acid sequence comprises, or alternatively consist essentially of, or yet further consist of, a nucleotide sequence encoding a CLN3 polypeptide, wherein the CLN3 polypeptide comprises SEQ ID NO.: 1, or an equivalent polypeptide, wherein an equivalent comprises an amino acid sequence having at least about 80%, at least about 85%, at least about 90%, at least about 95%, at least about 99%, or 100% amino acid sequence identity of SEQ ID NO 1. Additional non-limiting examples are provided in the attached sequence listing, incorporated herein by reference.

nucleic acid encoding CLN3 or an equivalent thereof can comprise, or alternatively consist essentially of, or yet further consist of, the polynucleotide of SEQ ID NO: 11, or a biological equivalent thereof. An example of a biological equivalent of CLN3 nucleic acid comprises a nucleic acid that hybridizes under conditions of high stringency to the complement of SEQ ID NO: 11 and encodes a protein having CLN3 biological activity. Another example includes a nucleic acid having at least 80% sequence identity to SEQ ID NO: 11 or its complement and encodes a protein having CLN3 biological activity. It is noted that human CLN3 variants are known in the art. Non-limiting examples include, GenBank Accession No: NM_001042432.1 (CLN3, transcript variant 1); GenBank Accession No: NM_001286104.1 (CLN3, transcript variant 3); GenBank Accession No: NM_001286105.1 (CLN3, transcript variant 4); GenBank Accession No: NM_001286109.1 (CLN3, transcript variant 5); GenBank Accession No: NM_001286110.1 (CLN3, transcript variant 6) or a nucleic acid having at least 80% sequence identity to any one of the examples listed above, or its complement, and encodes a protein having CLN3 biological activity.

In some embodiments, expression of the CLN3 transgene is driven by the retroviral Rous sarcoma virus (RSV) LTR promoter (optionally with the RSV enhancer). In some embodiments, expression of the CLN3 transgene is driven by the cytomegalovirus (CMV) promoter (optionally with the CMV enhancer). In some embodiments, expression of the CLN3 transgene is driven by the SV40 promoter. In some embodiments, expression of the CLN3 transgene is driven by the dihydrofolate reductase promoter. In some embodiments, expression of the CLN3 transgene is driven by the β-actin promoter. In some embodiments, expression of the CLN3 transgene is driven by the PGK promoter. In some embodiments, expression of the CLN3 transgene is driven by the EF1a promoter. In some embodiments, expression of the CLN3 transgene is driven by the UBC promoter. In some embodiments, expression of the CLN3 transgene is driven by the GUSB promoter. In some embodiments, expression of the CLN3 transgene is driven by the NSE promoter. In some embodiments, expression of the CLN3 transgene is driven by the Synapsin promoter. In some embodiments, expression of the CLN3 transgene is driven by MeCP2 promoter. In some embodiments, expression of the CLN3 transgene is driven by the GFAP promoter. In some embodiments, expression of the CLN3 transgene is driven by the CBh promoter. In preferred embodiments, expression of the CLN3 transgene is driven by the CBA promoter (e.g., SEQ ID NO: 24) or an equivalent thereof. In more preferred embodiments, expression of the CLN3 transgene is driven by the MeCP2promoter (e.g., nucleotides 775 to 1511 of SEQ ID No: 2, SEQ ID NO: 22 or SEQ ID NO: 23) or an equivalent of each thereof. In some embodiments, any one of the above mentioned promoters may be expressly excluded from driving expression of the CLN3 transgene.

Production of Recombinant AAV (rAAV)

In some embodiments, the AAV genome is from a naturally derived serotype or an isolate or a clade of AAV. In some embodiments, the AAV serotype is selected from the group of AAV1, AAV2, AAV4, AAV5, AAV6, AAV8, or AAV9. In some embodiments, the AAV serotype is AAV9. In other embodiments, the AAV serotype is AAV2.

Non-limiting examples include, the recombinant AAV can comprise, or alternatively consist essentially of, or yet further consist of, the polynucleotide of GenBank Accession No: AF063497.1 (AAV1, complete genome); the polynucleotide of GenBank Accession No: NC_001401.2 (AAV2, complete genome); the polynucleotide of GenBank Accession No: NC_001829 (AAV4, complete genome); the polynucleotide of GenBank Accession No: AX256321.1 (AAV5, synthetic construct); the polynucleotide of GenBank Accession No: AF028704.1 (AAV6, complete genome); the polypeptide of SEQ ID No: 35, the polynucleotide of SEQ ID No: 36, or a biological equivalent thereof. Another example includes a nucleic acid having at least 80% sequence identity to the above referenced polynucleotides.

A suitable recombinant adeno-associated virus (AAV) can be generated by culturing a host cell that contains a nucleic acid sequence encoding an adeno-associated virus (AAV) serotype capsid protein, or fragment thereof, a functional rep gene; a minigene composed of, at a minimum, AAV inverted terminal repeats (ITRs) and the nucleic acid sequence encoding the CLN3 polypeptide (or variants or equivalents thereof as described herein), and sufficient helper functions to permit packaging of the minigene into the AAV capsid protein.

The components to be cultured in the host cell to package an AAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, in certain embodiments, the required component(s) may be under the control of a constitutive promoter.

In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV may be delivered to the packaging host cell using any appropriate genetic element (vector). The selected genetic element may be delivered by any suitable method, including those described herein. Such methods are known to those with skill in the art (see, e.g., Sambrook et al, Molecular Cloning: A Laboratory Manual, Cold Spring Harbor Press, Cold Spring Harbor, N.Y.). Similarly, methods of generating rAAV virions are well known (see, e.g., Fisher et al. (1993) J. Virol., 70:520-532; U.S. Pat. No. 5,478,745; and the like).

In one illustrative, but non-limiting embodiment, rAAVs may be produced using the triple transfection method (e.g., as described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Typically, the AAV helper function vector supports efficient AAV vector production without generating any detectable wild-type AAV virions (i.e., AAV virions containing functional rep and cap genes). Illustrative, but non-limiting examples of vectors include pHLP19 (see U.S. Pat. No. 6,001,650), pRep6cap6 vector (see, e.g. U.S. Pat. No. 6,156,303), and the like. The accessory function vector can encode nucleotide sequences for non-AAV derived viral and/or cellular functions upon which AAV is dependent for replication (i.e., “accessory functions”). The accessory functions can include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

### Recombinant AAV Administration

The rAAVs are typically administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected tissue (e.g., intracerebral administration, intrathecal administration), intravenous, oral, aerosol administration, administration via inhalation (including intranasal and intratracheal delivery), intravenous, intramuscular, subcutaneous, intradermal, rectal, and other parental routes of administration. Routes of administration may be combined, if desired.

In certain embodiments delivery of certain rAAVs to a subject may be, for example, by administration into the bloodstream of the subject. Administration into the bloodstream may be by injection into a vein, an artery, or any other vascular conduit. Moreover, in certain instances, it may be desirable to deliver the rAAVs to brain tissue, meninges, neuronal cells, glial cells, astrocytes, oligodendrocytes, cerebrospinal fluid (CSF), interstitial spaces and the like. In some embodiments, recombinant AAVs may be delivered directly to the spinal cord or brain by injection into the ventricular region, as well as to the striatum (e.g., the caudate nucleus or putamen of the striatum), and neuromuscular junction, or cerebellar lobule, with a needle, catheter or related device, using neurosurgical techniques known in the art, such as by stereotactic injection (see, e.g., Stein et al. (1999) J Virol. 73:3424-3429; Davidson et al. (2000) Proc. Natl. Acad. Sci. USA; Davidson et al. (1993) Nat. Genet. 3:219-223; Alisky and Davidson (2000) Hum. Gene Ther. 11:2315-2329; and the like).

Methods for delivering a transgene to central nervous system (CNS) tissue in a subject are provided herein. The methods typically involve administering to a subject an effective amount of a rAAV comprising a nucleic acid vector for expressing a transgene (e.g., CLN3) in the subject. An effective amount refers to an amount that is capable of treating or ameliorating juvenile neuronal ceroid lipofuscinosis (JNCL or Juvenile Batten disease) and/or reducing, suppressing, inhibiting, lessening, ameliorating or affecting the progression, severity, and/or scope of the disease or condition.

The effective amount will depend on a variety of factors such as, for example, the species, age, weight, health of the subject, and the CNS tissue to be targeted, and may thus vary among subject and tissue. An effective amount may also depend on the mode of administration. For example, targeting a CNS tissue by intravascular injection may require different (e.g., higher) doses, in some cases, than targeting CNS tissue by intrathecal or intracerebral injection. In some cases, multiple doses of a rAAV are administered while in other cases, as illustrated in the examples herein, a single dose can be sufficient. An effective amount may also depend on the rAAV used. For example, dosages for targeting a CNS tissue may depend on the serotype (e.g., the capsid protein) of the rAAV. For example, the rAAV may have a capsid protein of an AAV serotype selected from the group consisting of: AAV1, AAV2, AAV5, AAV6, AAV6.2, AAV7, AAV8, AAV9, rh. 10, rh.39, rh.43 and CSp3. In certain embodiments, the effective amount of rAAV is 10¹⁰, 10¹¹, 10¹², 10¹³, or 10¹⁴, or 10¹⁵, or 10¹⁶ genome copies per kg. In certain embodiments, the effective amount of rAAV is 10¹⁰, 10¹¹, 10¹², 10¹³, or 10¹⁴, or 10¹⁵, or 10¹⁶ genome copies per subject. When multiple doses are administered, the same or different serotype can be combined for the therapy.

A method for delivering a transgene to CNS tissue in a subject may comprise administering a rAAV by a single route or by multiple routes. For example, delivering a transgene to CNS tissue in a subject may comprise administering to the subject, by intravenous administration, an effective amount of a rAAV that crosses the blood-brain-barrier. Delivering a transgene to CNS tissue in a subject may comprise administering to the subject an effective amount of a rAAV by intrathecal administration or intracerebral administration, e.g., by intraventricular injection. A method for delivering a transgene to CNS tissue in a subject may comprise co-administering of an effective amount of a rAAV by two different administration routes, e.g., by intrathecal administration and by intracerebral to administration. Co-administration may be performed at approximately the same time, or different times.

In certain embodiments the CNS tissue to be targeted may be selected from cortex, hippocampus, thalamus, hypothalamus, cerebellum, brain stem, cervical spinal cord, thoracic spinal cord, and lumbar spinal cord, for example. The administration route for targeting CNS tissue typically depends on the AAV serotype as discussed above.

In certain embodiments, it may be desirable to first perform diagnostic confirmation of a presumed subject (or for carrier or prenatal testing) to identify mutations in the CLN3 gene prior to administering to the subject an effective amount of a AAV comprising a nucleic acid vector for expressing a transgene (e.g., CLN3) in the subject. Such testing can, for example, be done by PCR amplification and sizing analysis for the common 1 kb deletion or by full sequencing analysis to cover the 15 exons of the CLN3 gene, as well as adjacent intronic regions. Sequencing can uncover any nonsense, missense, splicing mutations, small insertion and deletion mutations which have all been reported in the CLN3 gene.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intracerebrally, intrathecally, intracerebrally, orally, intraperitoneally, or by inhalation. In some embodiments, the administration modalities as described in U.S. Pat. Nos. 5,543,158; 5,641,515 and 5,399,363 may be used to deliver rAAVs.

In some embodiments, the vectors, recombinant adeno-associated virus, pharmaceutical formulations, and the like described herein can be administered with an additional therapeutic. Non-limiting examples of additional therapeutics include, mycophenolate mofetil, carbenoxolene, glycyrrhetinic acid, glycyrrhizic acid, chaperone therapy, enzyme replacement therapy, stem cell therapy, anticonvulsant medications, antioxidant supplementation (e.g., vitamin C, vitamin E, methionine, B6), omega-3 fatty acids. (see, e.g., Hobert et al., (2006) Biochimica et Biophysica Acta 1762 (10): 945-953). Dosage, treatment protocol, and routes of administration for additional therapeutics are known in the art and/or within the ability of a skilled clinician to determine, based on the type of treatment, etc.

In one embodiment, the vectors, recombinant adeno-associated virus, pharmaceutical formulations, and the like described herein and the additional therapeutic are administered sequentially. In another embodiment, the vectors, recombinant adeno-associated virus, pharmaceutical formulations, and the like described herein and the additional therapeutic are administered simultaneously. In one embodiment, the vectors, recombinant adeno-associated virus, pharmaceutical formulations, and the like described herein are administered after the period of time of administration of an additional therapeutic. In a further aspect, the rAAV therapy is co-administered with other complimentary therapy to alleviate or treat the symptoms of disease.

### Recombinant AAV Compositions

In certain embodiments the rAAVs may be delivered to a subject in compositions according to any appropriate methods known in the art. For example, the rAAV, suspended in a physiologically compatible carrier (e.g., in a composition), may be administered to a subject, e.g., a human, mouse, rat, cat, dog, sheep, rabbit, horse, cow, goat, pig, guinea pig, hamster, chicken, turkey, or a non-human primate (e.g., Macaque). The compositions may comprise a rAAV alone, or in combination with one or more other viruses (e.g., a second rAAV encoding having one or more different transgenes). In some embodiments, a compositions comprise 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other illustrative carriers include, but are not limited to, sterile saline, lactose, sucrose, calcium phosphate, gelatin, dextran, agar, pectin, peanut oil, sesame oil, and water.

Optionally, the compositions of the invention may contain, in addition to the rAAV and carrier(s), other conventional pharmaceutical ingredients, such as preservatives, or chemical stabilizers. Suitable illustrative preservatives include, but are not limited to chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some embodiments, rAAV compositions are formulated to reduce aggregation of AAV particles in the composition, particularly where high rAAV concentrations are present (e.g., about 10¹³ vg/ml or more). Methods for reducing aggregation of rAAVs are well known in the art and, include, for example, addition of surfactants, pH adjustment, salt concentration adjustment, etc. (see, e.g., Wright et al. (2005) Mol. Ther. 12:171-178; and the like).

Formulation of pharmaceutically-acceptable excipients and carrier solutions is well-known to those of skill in the art, as is the development of suitable dosing and treatment regimens for using the particular compositions described herein in a variety of treatment regimens. Typically, these formulations may contain at least about 0.1% of the active ingredient or more, although the percentage of the active ingredient(s) may, of course, be varied and may conveniently be, for example, between about 1 or 2% and about 70% or 80% or more of the weight or volume of the total formulation. Naturally, the amount of active ingredient in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The pharmaceutical forms suitable for injectable use typically include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. The formulation is typically stable under the conditions of manufacture and storage and preserved against the contaminating action of microorganisms, such as bacteria and fungi. In certain embodiments the carrier can be a solvent or dispersion medium containing, for example, water, ethanol, polyol (e.g., glycerol, propylene glycol, and liquid polyethylene glycol, and the like), suitable mixtures thereof, and/or vegetable oils. In certain embodiments proper fluidity may be maintained, for example, by the use of a coating, such as lecithin, by the maintenance of the required particle size in the case of dispersion and by the use of surfactants. In various embodiments the prevention of the action of microorganisms can be brought about by various antibacterial and antifungal agents, for example, parabens, chlorobutanol, phenol, sorbic acid, thimerosal, and the like. In many cases, it will be desirable to include isotonic agents, for example, sugars or sodium chloride. Prolonged absorption of the injectable compositions can be brought about by the use in the compositions of agents delaying absorption, for example, aluminum monostearate and gelatin.

In certain embodiments for administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see, e.g., “Remington's Pharmaceutical Sciences” 15th Edition, pages 1035-1038 and 1570-1580). Some variation in dosage may occur depending on the condition of the host. In some embodiments, the person responsible for administration will, in any event, determine the appropriate dose for the individual host.

In certain embodiments sterile injectable solutions can be prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, illustrative methods of preparation include, but are not limited to vacuum-drying and freeze-drying techniques which yield a powder of the active ingredient plus any additional desired ingredient from a previously sterile-filtered solution thereof.

In certain embodiments the rAAV compositions disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present invention into suitable host cells. In particular, the rAAV vector delivered trans genes may be formulated for delivery either encapsulated in a lipid particle, a liposome, a vesicle, a nanosphere, or a nanoparticle or the like.

In certain embodiments such formulations may be used pharmaceutically acceptable formulations of the nucleic acids or the rAAV constructs disclosed herein. The formation and use of liposomes is generally known to those of skill in the art (see, e.g., U.S. Pat. Nos. 5,741,516; 5,567,434; 5,552,157; 5,565,213; 5,738,868; 5,795,587; and the like).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 angstrom., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) can be designed using polymers able to be degraded in vivo. In certain embodiments biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations, transdermal matrices (U.S. Pat. Nos. 5,770,219 and 5,783,208) and feedback-controlled delivery (U.S. Pat. No. 5,697,899).

### Kits and Related Compositions

The agents described herein may, in some embodiments, be assembled into pharmaceutical or diagnostic or research kits to facilitate their use in therapeutic, diagnostic or research applications. A kit may include one or more containers housing the components of the invention and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

The kit may be designed to facilitate use of the methods described herein and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit.

As used herein, “instructions” can define a component of instruction and/or promotion, and typically involve written instructions on or associated with packaging of the invention. Instructions also can include any oral or electronic instructions provided in any manner such that a user will clearly recognize that the instructions are to be associated with the kit, for example, audiovisual (e.g., videotape, DVD, etc.), internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to a subject, such as a syringe, topical application devices, or IV needle tubing and bag.

The therapies as describe herein can be combined with appropriate diagnostic techniques to identify and select patients for the therapy. For example, a sample is isolated from a patient and the sample is tested for a mutation that has been correlated to development of Juvenile Batten Disease (homozygous or heterozygous). Thus, patients harboring the mutation can be identified prior to symptoms appearing or before advancement of the disease.

### EXAMPLES

The following examples are offered to illustrate, but not to limit the claimed invention.

### Example 1

### Gene Therapy for the Treatment of Juvenile Batten Disease

This example describes initial studies of gene therapy for the treatment of Juvenile Batten Disease. While gene therapy has shown promise in correcting forms of Batten Disease that involve mutations in soluble enzymes, in part, due to cross-correction of neighboring non-transduced cells (Passini et al. (2006) J. Neurosci. 26 (5): 1334-1342; Sondhi et al. (2012) Hum. Gene Ther. Meth. 23 (5): 324-335; Macauley et al. (2014) J. Neurosci. 34 (39): 13077-13082), such approaches have been viewed as problematic for the treatment of JNCL because CLN3 is a transmembrane protein (Cotman et al. (2012) Clin. Lipidol. 7 (1): 79-91), meaning more cells must be transduced to produce a phenotypic effect. However, the data provided herein demonstrate that it is possible to deliver CLN3 to enough cells to improve motor behavior in a mouse model of JNCL.

Low CLN3 expression levels were driven with the promoter for methyl-CpG binding protein 2 (MeCP2) that was previously effective in a dosage-dependent model of Rett syndrome (Garg et al. (2013) J. Neurosci. 33 (34): 13612-13620), an autism spectrum disorder, with a separate construct driving high levels of CLN3 via the β-actin promoter. The rationale for this approach was based on the prediction that low CLN3 levels are required for normal cellular function, since postnatal CLN3 expression is limited (Eliason et al. (2007) J. Neurosci. 27 (37): 9826-9834). This study represents the first demonstration of a CLN3 dosage effect in vivo, where it was surprisingly observed that high levels of CLN3 overexpression masked a therapeutic benefit.

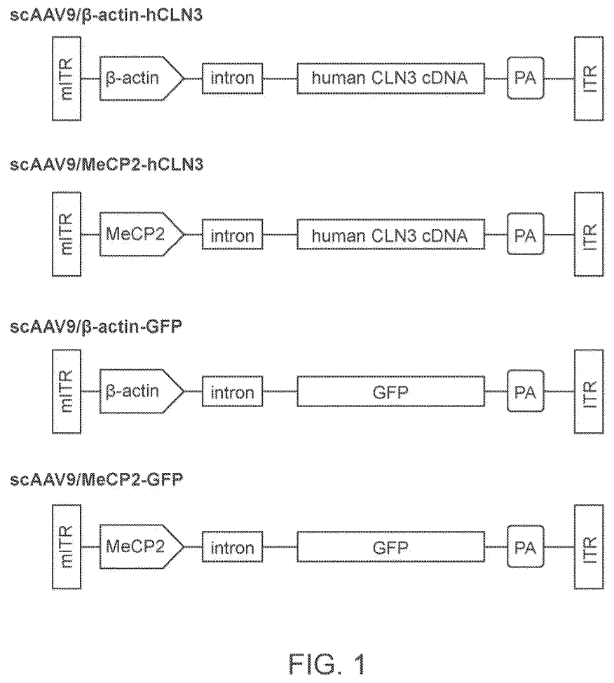

As described below, for proof-of-principle studies, 1 month-old CLN3^{Δex7/8} mice received one dose of 2×10¹² viral genomes (vg) of self-complementary (sc) AAV9/β-actin-hCLN3 (high expressing) or scAAV9/MeCP2-hCLN3 (low expressing) intravenously, with viruses driving green fluorescence protein (GFP) expression (scAAV9/β-actin-GFP and scAAV9/MeCP2-GFP) as controls (see, FIG. 1). Importantly, a promoter-dosage effect for hCLN3 expression was confirmed in several brain regions where neurons are destined to die in JNCL, including the hippocampus (HPC), striatum (STR), thalamus (TH), visual cortex (VC), and cerebellum (CB), where hCLN3 levels were elevated from 3- to 8-fold in CLN3^{Δex7/8} mice receiving scAAV9/β-actin-hCLN3 vs. scAAV9/MeCP2-hCLN3.

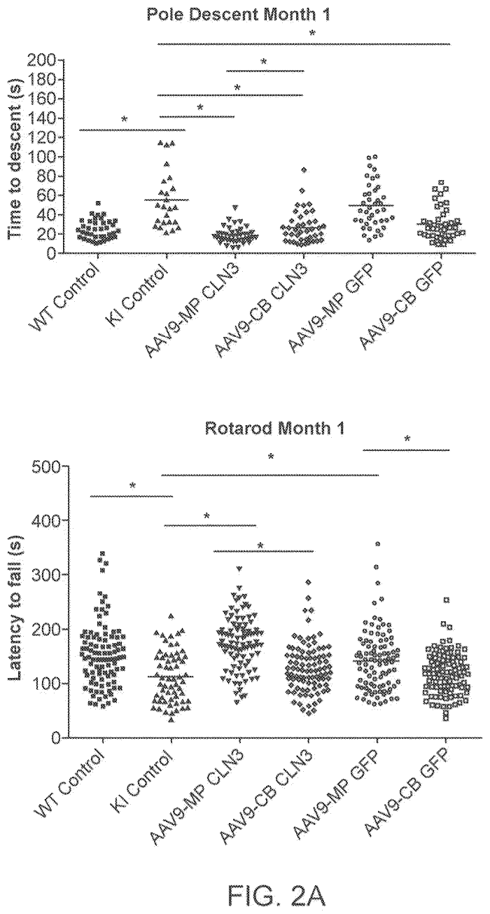

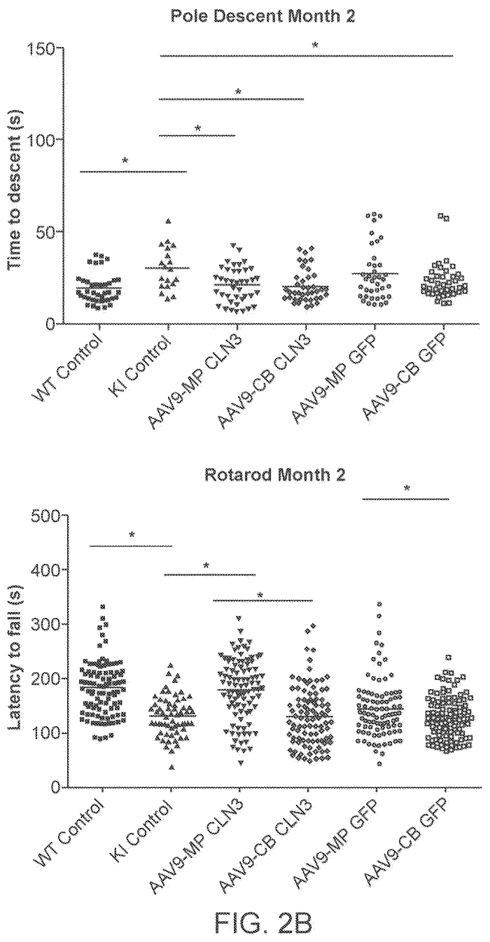

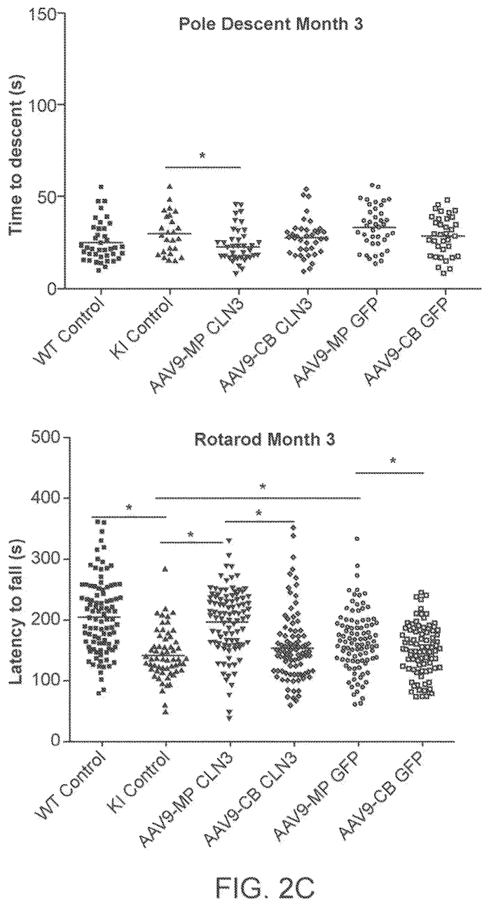

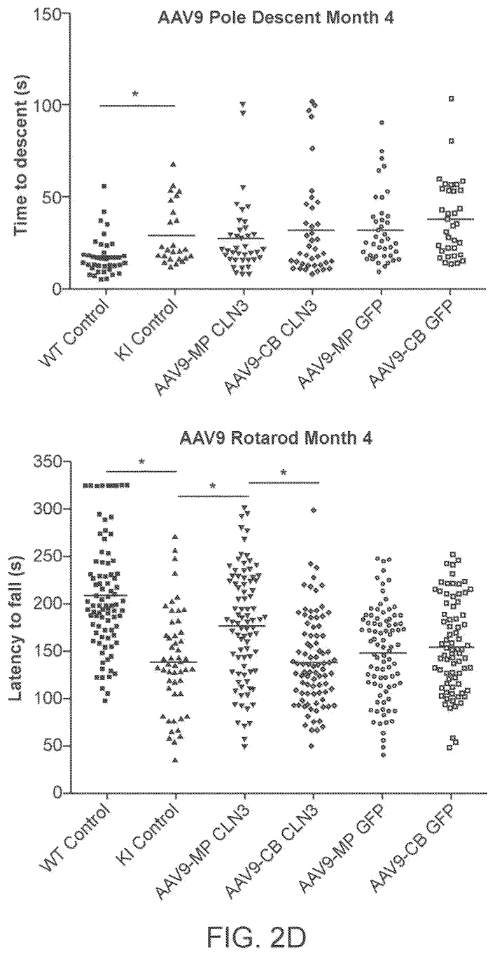

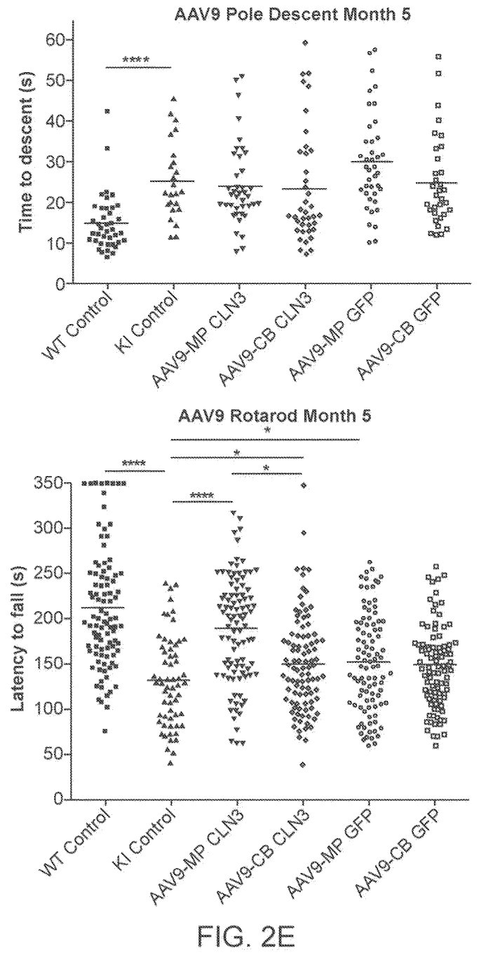

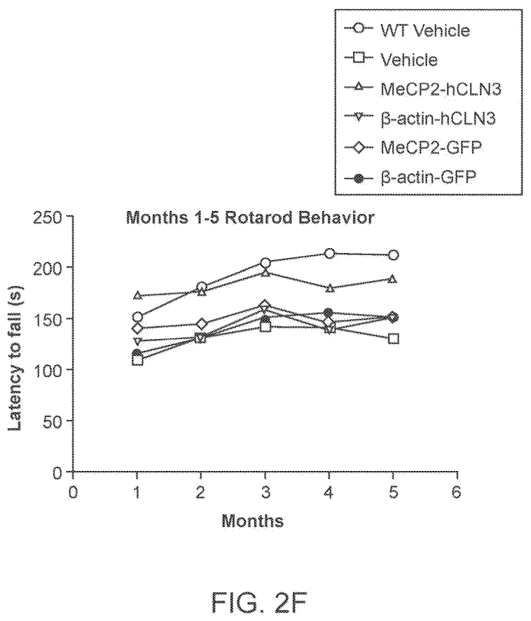

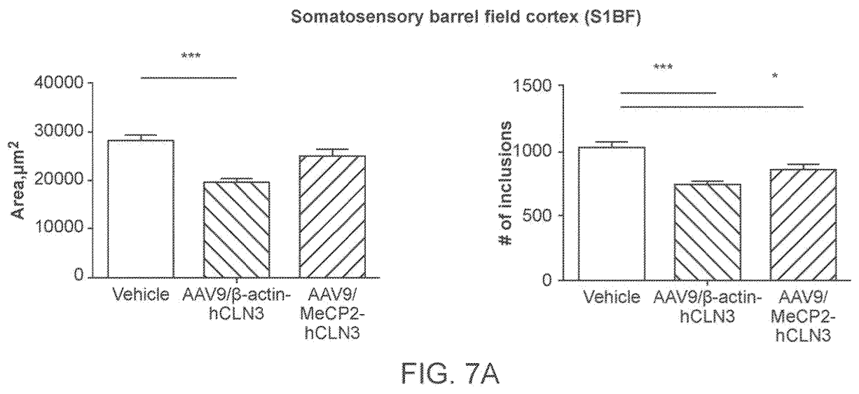

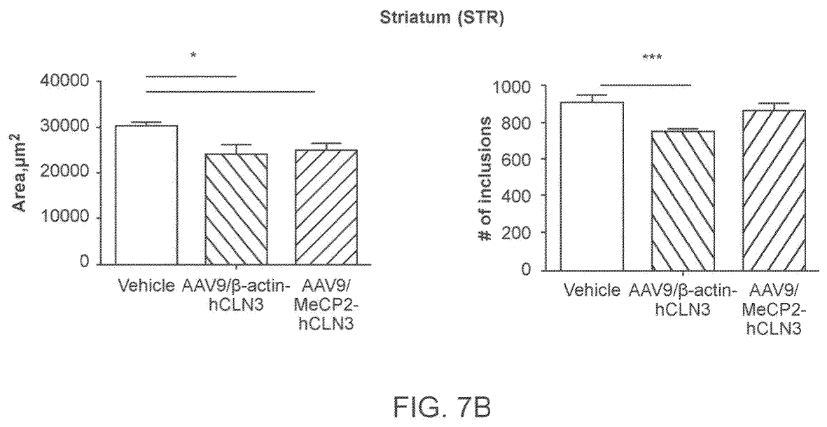

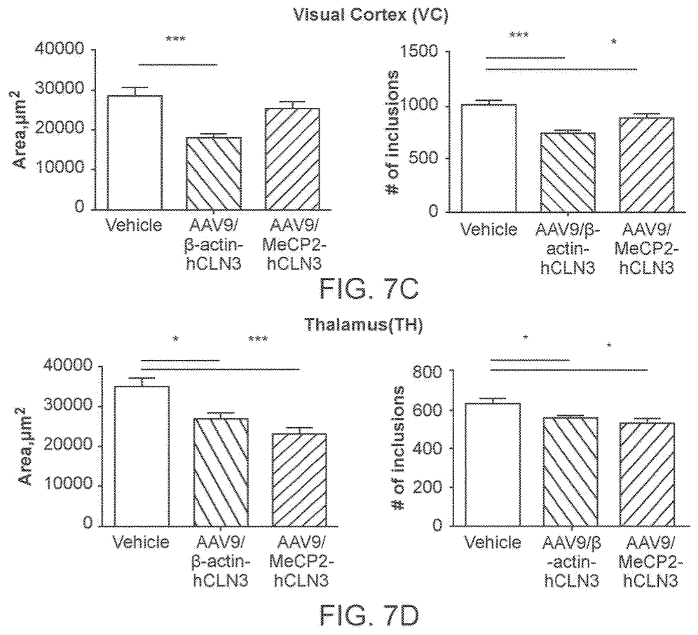

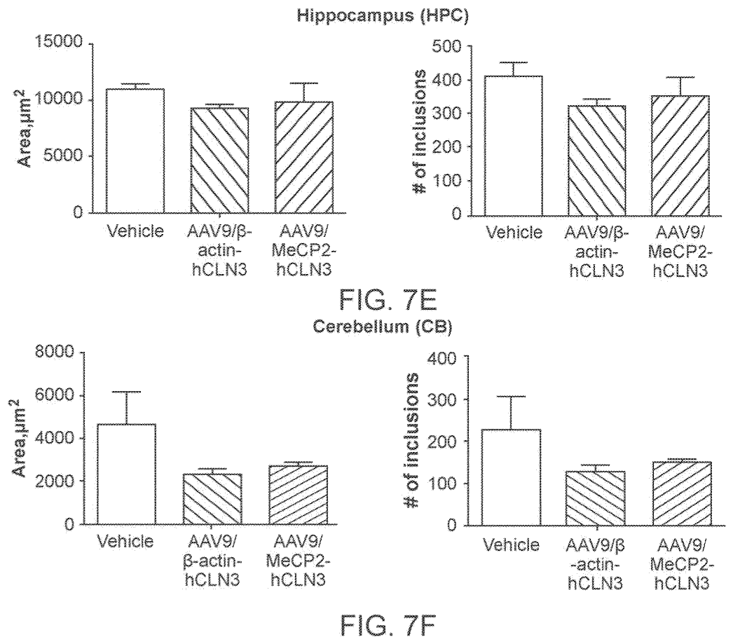

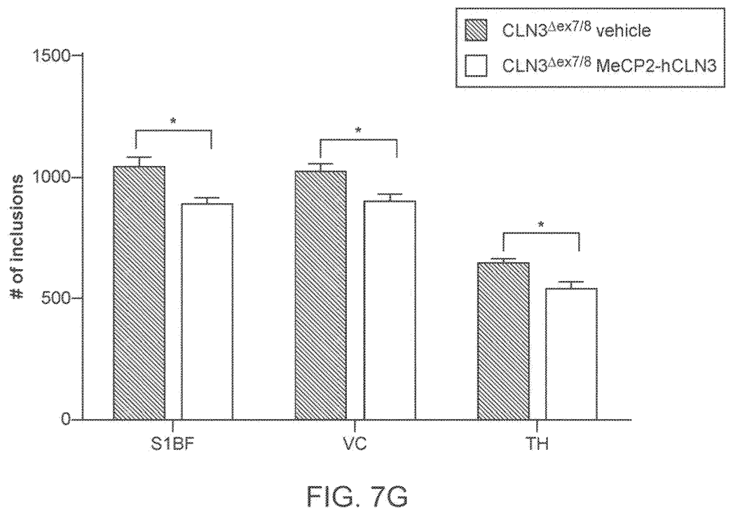

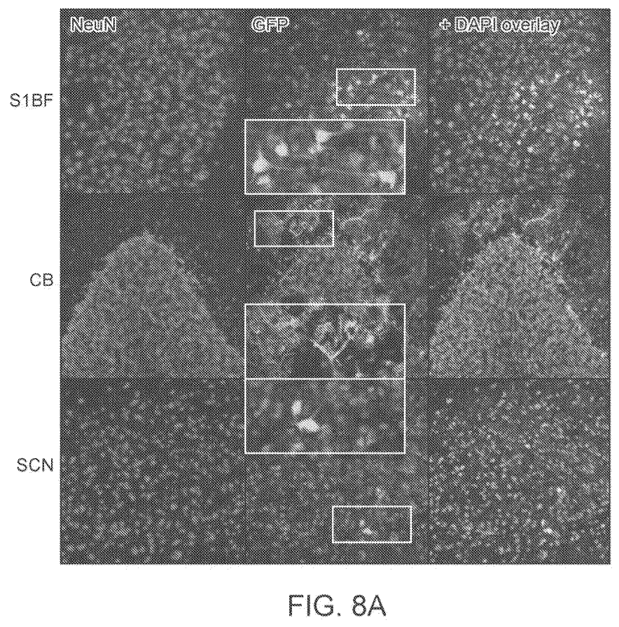

However, there was a disconnect between hCLN3 levels and neuroprotection, since only the scAAV9 construct driving low hCLN3 expression (scAAV9/MeCP2-hCLN3) was capable of correcting motor deficits in CLN3^{Δex7/8} mice (accelerating rotarod and pole descent), demonstrating the surprising result that high CLN3 expression in the brain was not advantageous for improved motor coordination. (see, FIG. 2A-2E). Similar benefits were observed with lysosomal storage material, which was significantly reduced in the somatosensory barrel field cortex (SIBF) (see, FIG. 7A), VC (see, FIG. 7C), and TH (see, FIG. 7D) of animals receiving scAAV9/MeCP2-hCLN3.

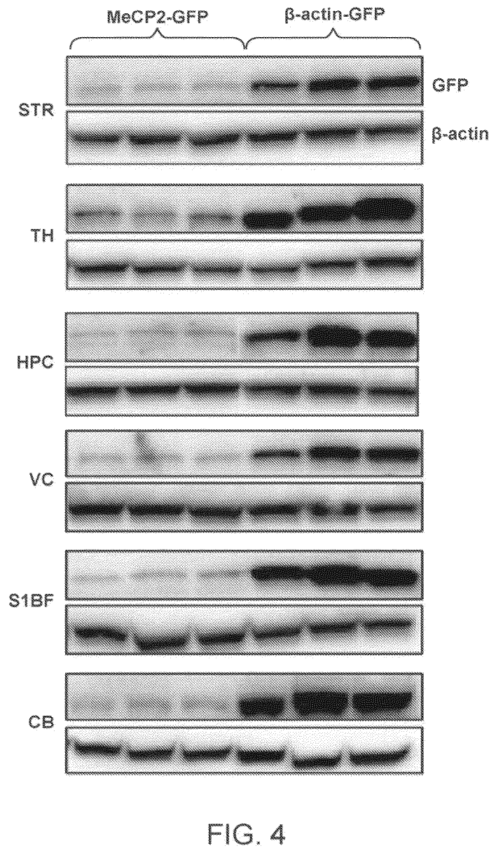

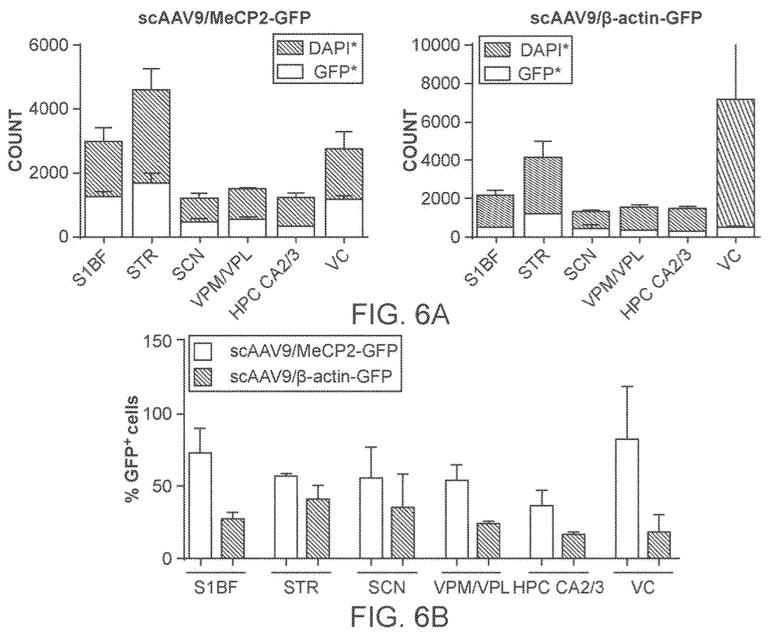

The beneficial effects of scAAV9/MeCP2-hCLN3 on motor behavior in CLN3^{Δex7/8} mice were not observed with scAAV9/GFP control constructs, confirming specificity of action for hCLN3 (see, FIG. 2A-2E). The extent of viral transduction in the brain was widespread, as evident by GFP expression in the SIBF, VC, STR, TH, HPC, and CB (see, FIG. 4). scAAV9/MeCP2-hCLN3 transduced NeuN neurons and GFAP astrocytes in 1 month-old CLN3^{Δex7/8} mice, whereas limited expression was detected in microglia (see, FIG. 6A-6C). Although wild type CLN3 expression was only restored in a subset of neurons and glia, this was sufficient to correct disease-associated pathology in CLN3^{Δex7/8} animals (as measured by improved motor function and decreased lysosomal storage material). Without wishing to be bound by theory, it is contemplated that this may reflect the redundancy of cells in behavioral circuits or compensation by other mechanisms to supplant circuits weakened by insufficient neuronal/glial CLN3, and/or may reflect an underestimate in the number of cells that are transduced.

Based on these observations, it is believed that scAAV9/MeCP2-hCLN3 gene therapy will target sufficient numbers of CNS cells to significantly improve JNCL outcome.

### Methods and Results

Self-complementary AAV9 (scAAV9) persists as a stable episome in non-dividing cells with studies reporting stable transgene expression for years. scAAV9 vectors are 10- to 100-fold more efficient than traditional single-stranded (ss) AAV vectors (McCarty et al. (2003) Gene Ther. 10 (26): 2112-2118; McCarty et al. (2001) Gene Ther. 8 (16): 1248-1254), but can only package foreign DNA <2.2 kb, which was not a limitation for the approach described herein, since the hCLN3 cDNA is only 1.3 kb. The scAAV9 constructs were engineered to harbor hCLN3 to facilitate its translational potential (see, e.g., FIG. 1). In certain constructs the methylCpG binding protein 2 (MeCP2) promoter was selected to drive hCLN3 expression.

The feasibility of a gene therapy approach for JNCL is supported by data showing systemic i.v. delivery of scAAV9/MeCP2-hCLN3 (single dose) into one month-old CLN3^{Δex7/8} mice significantly improved motor behavior, which was not observed with control viruses harboring GFP (FIG. 2A). Improved motor behavior continued to be observed at 2 months post-transduction (FIG. 2B), at 3 months post-transduction (FIG. 2C), at 4 months post-transduction (FIG. 2D), and at 5 months post-transduction (FIG. 2E). Analysis of blood chemistry profiles at 1 month and at 3 months revealed no abnormalities in any treatment group (data not shown).

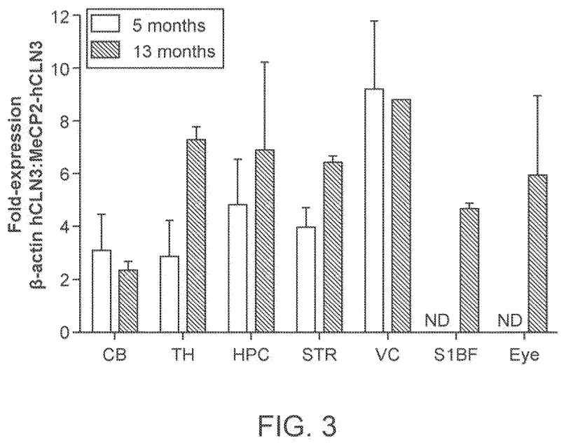

scAAV9 constructs were engineered to achieve high versus low hCLN3 expression in transduced cells using the β-actin and MeCP2 promoters, respectively. 1 month old CLN3^{Δex7/8} mice were transduced with virus via the retro-orbital sinus. Mice were sacrificed at 5 months and 13 months post-transduction, mice were 6 months and 14 months old, respectively. Brain regions were dissected from vibratome sections (300 μm thick). A promoter dosage effect was confirmed for both hCLN3, where expression was elevated from approximately 3- to 8-fold in the cerebellum (CB), thalamus (TH), hippocampus (HPC), striatum (STR), visual cortex (VC), somatosensory barrel field cortex (SIBF), and the eye of CLN3^{Δex7/8} mice treated with scAAV9/β-actin-hCLN3 compared to the low expressing scAAV9/MeCP2-hCLN3 (FIG. 3), and GFP protein (FIG. 4).

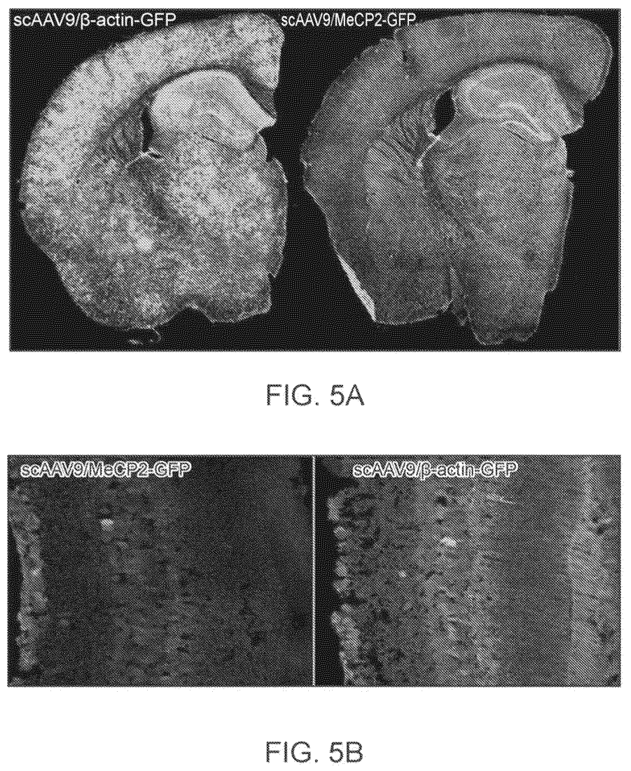

In addition, confocal images were acquired from brain cross sections collected from mice 5 months post-transduction to further visualize and confirm distribution and expression levels. Images were acquired using the same confocal settings to highlight expression differences. Results show wide biodistribution in both CLN3^{Δex7/8} mice treated with scAAV9/β-actin-hCLN3 and scAAV9/MeCP2-hCLN3, with GFP intensity greater in the scAAV9/β-actin-hCLN3 treated mice. FIG. 5A. Results also showed distribution of both scAAV9/β-actin-hCLN3 and scAAV9/MeCP2-hCLN3 in retinal regions of mice 13 months following transduction. FIG. 5B.

Comparative biodistribution of scAAV9/β-actin-GFP or scAAV9/MeCP2-GFP in the brains of CLN3^{Δex7/8} mice 5 months after administration of scAAV9/β-actin-hCLN3 and scAAV9/MeCP2-hCLN3 was determined by counting GFP cells and total cell number (DAPI). FIG. 6A. As summarized in FIG. 6B, scAAV9/MeCP2-hCLN3 treated mice displayed a significant increase in the percentage of GFP cells in the SIBF, VPM/VPL, and VC regions of the brain.

The surprising finding of promoter dosage effect was further substantiated by similarities in the percentages of NeuN and GFAP cells transduced by both promoter constructs (data not shown). Importantly, the data show that CLN3^{Δex7/8} mice receiving a single injection of the low expressing construct (scAAV9/MeCP2-hCLN3) displayed consistent improvements in motor coordination out to five months post-transduction (latest interval examined to date) whereas the high expressing construct (scAAV9/β-actin-hCLN3) was not effective (FIGS. 2A-2E).

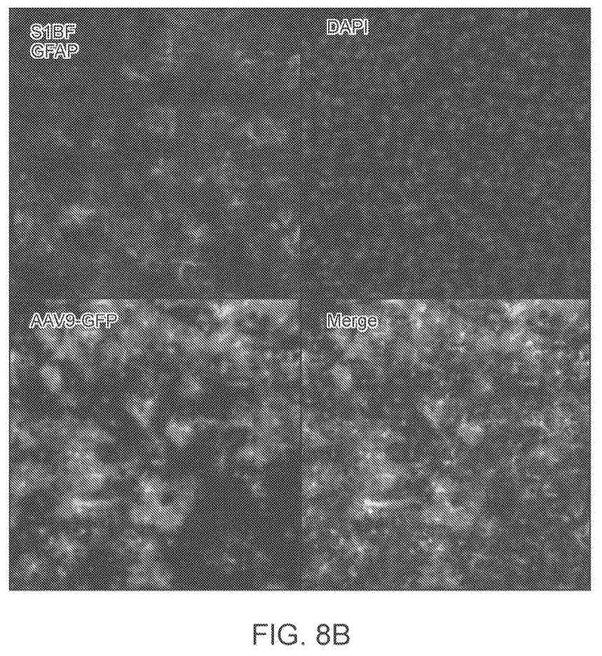

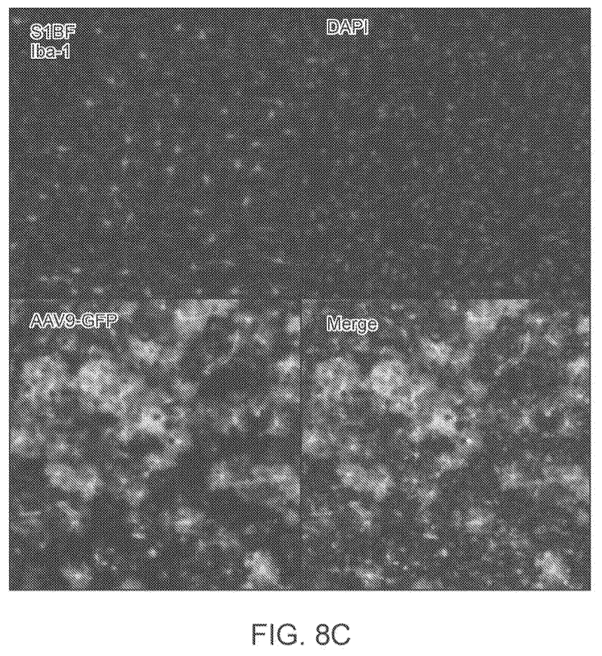

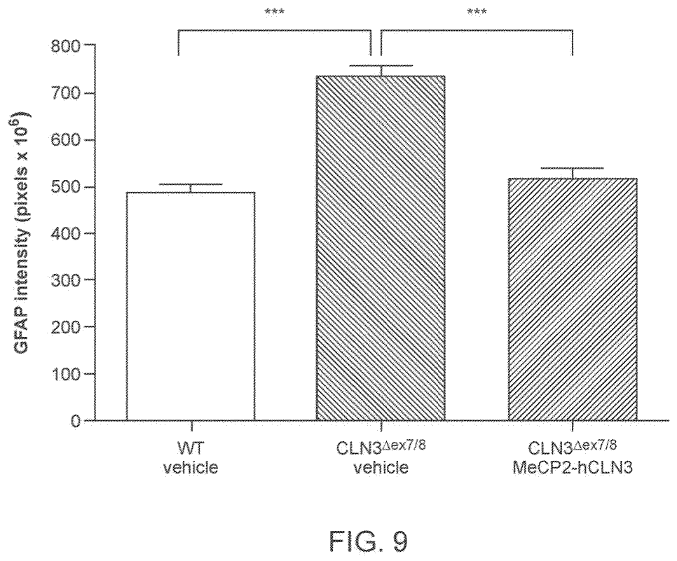

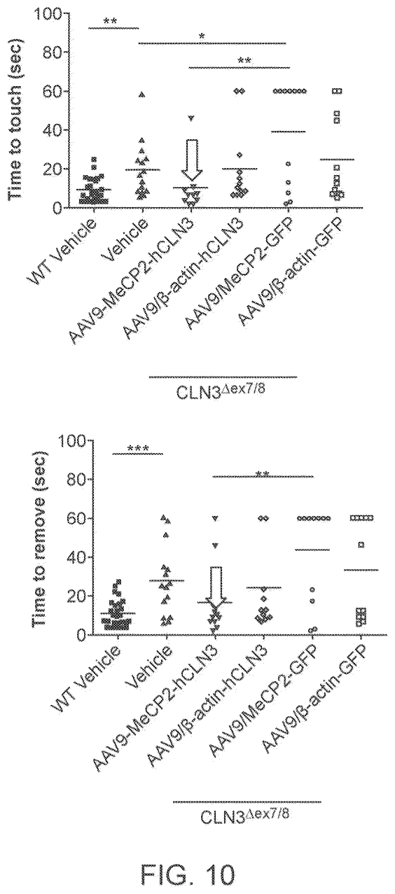

In addition, significant reductions in lysosomal storage material were observed in the brains of CLN3^{Δex7/8} mice receiving scAAV9/MeCP2-hCLN3 (FIGS. 7A-7G). The biodistribution of scAAV9 was widespread, with NeuN⁺ neurons and GFAP⁺ astrocytes transduced in the SIBF, VC, HPC, STR, TH, CB, and suprachiasmatic nucleus (SCN; FIG. 8A and data not shown), in agreement with other reports using scAAV9 in models of Rett syndrome and spinal muscular atrophy (SMA) (Garg et al. (2013) J. Neurosci. 33 (34): 13612-13620; Foust et al. (2010) Nat. Biotechnol. 28 (3): 271-274). As shown in FIG. 8B systemic administration of scAAV9 transduces astrocytes in the CLN3^{Δex7/8} brain. FIG. 8C shows that systemic administration of scAAV9 does not transduce microglia in the CLN3^{Δex7/8} brain.

Although, it appears that only a fraction of CNS cells were transduced by the virus, the behavioral improvements in CLN3^{Δex7/8} animals suggest beneficial intrinsic effects of hCLN3 in neurons and perhaps non-cell autonomous contributions from hCLN3 transduced glia. The latter would be expected to promote neuron survival based on the well appreciated role of astrocytes in regulating glutamate levels and neuron activity at the tripartite synapse (Perea et al. (2009) Trends Neurosci. 32 (8): 421-431).

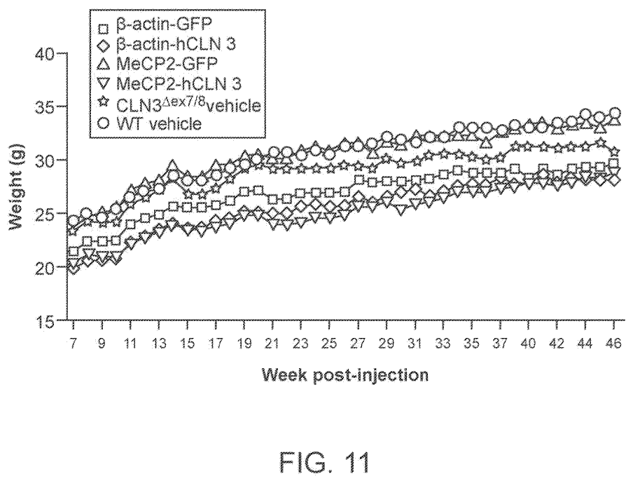

Another common symptom to JNCL is weight loss. The beneficial effects of scAAV9/MeCP2-hCLN3 on weight gain in CLN3^{Δex7/8} mice were not observed with the scAAV9/β-actin-hCLN3 or scAAV9/GFP control constructs confirming specificity of action for hCLN3 (FIG. 11).

It is noted that the approach described herein is advantageous over conventional therapies in that it can employ a systemic delivery route to enhance virus biodistribution and a unique promoter (MeCP2) to drive low CLN3 expression, since the preliminary data shows that high CLN3 levels are ineffective. In summary, several attributes of scAAV9/MeCP2-hCLN3 bolster the likelihood of success for improving JNCL outcome, namely; 1) this approach will correct the genetic defect at the root cause of the disease; 2) the virus can efficiently transduce CNS cells in juvenile and adult animals, which is critical because JNCL is not diagnosed until 5-10 years of age; 3) the MeCP2 promoter drives low level hCLN3 expression, which the data presented herein shows is essential for improving motor function in CLN3^{Δex7/8} mice; and 4) constructing the virus to harbor hCLN3 will accelerate its translation to clinical trials.

### Example 2

### Safety, Toxicity and Inflammation Assessment

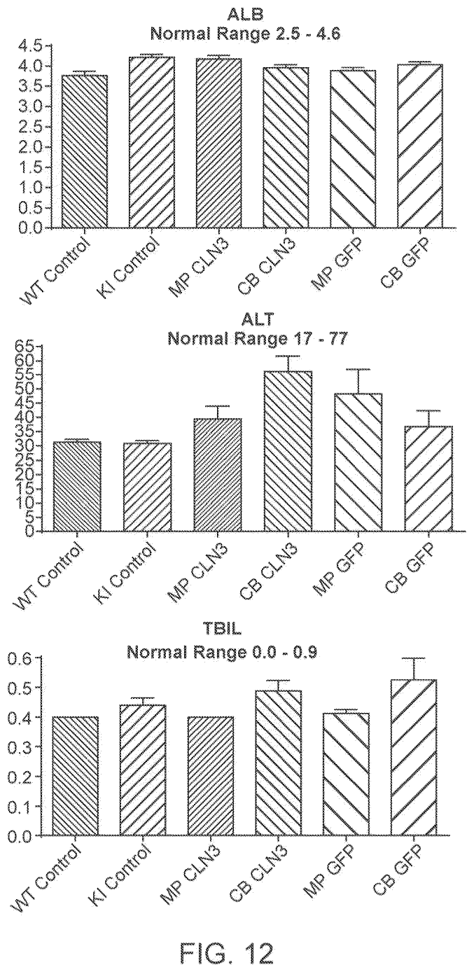

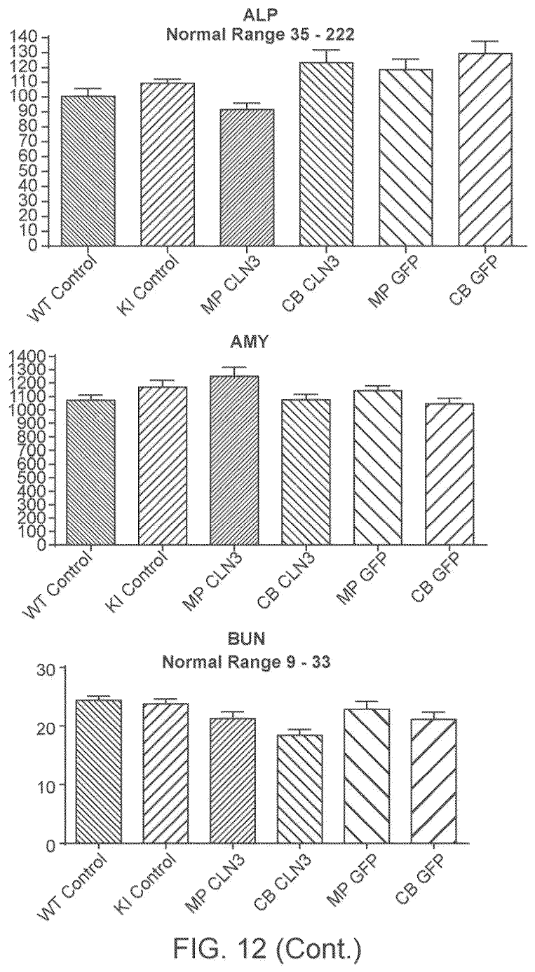

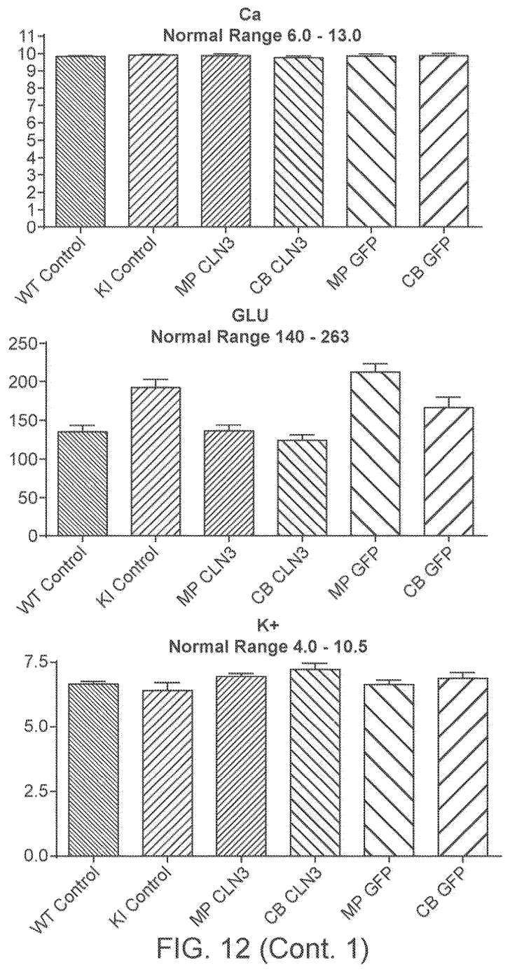

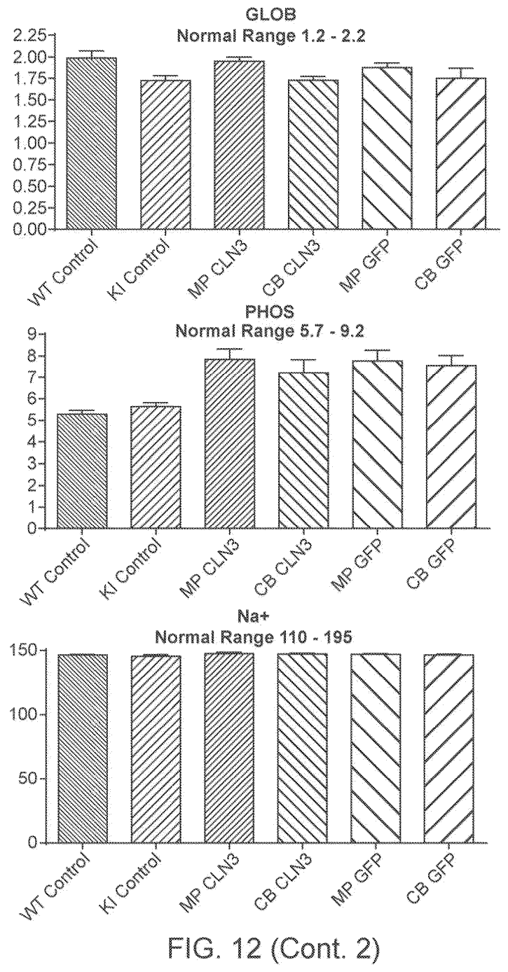

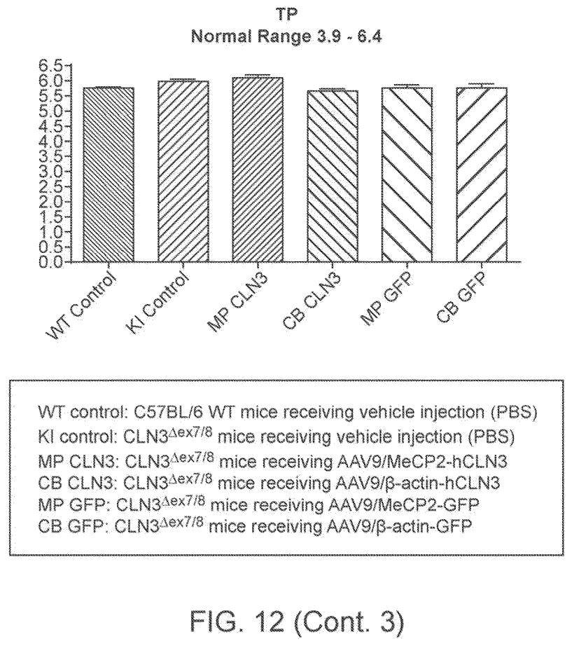

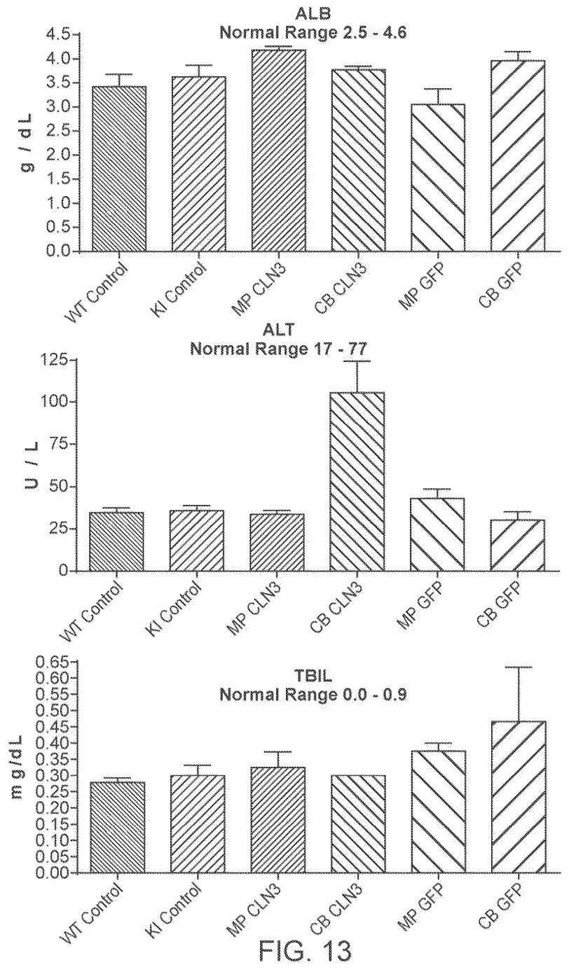

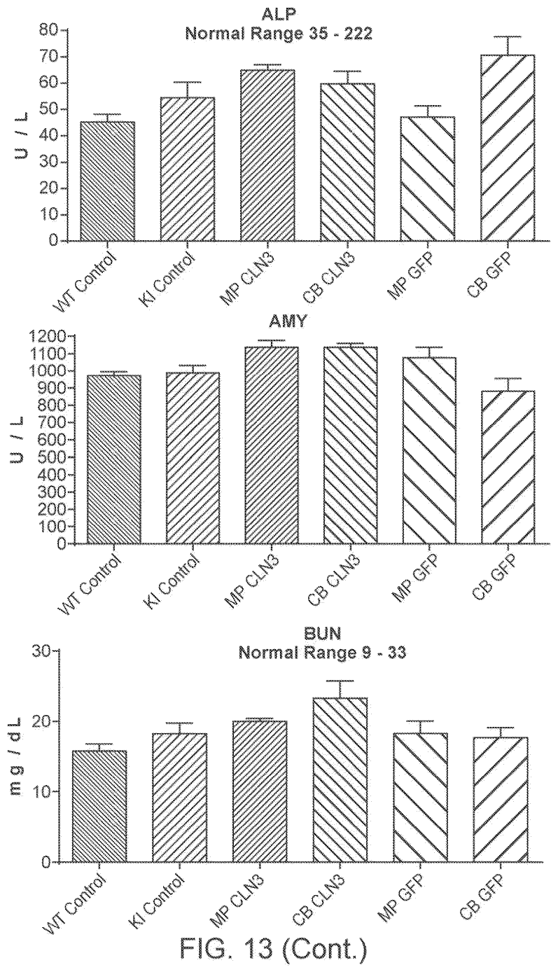

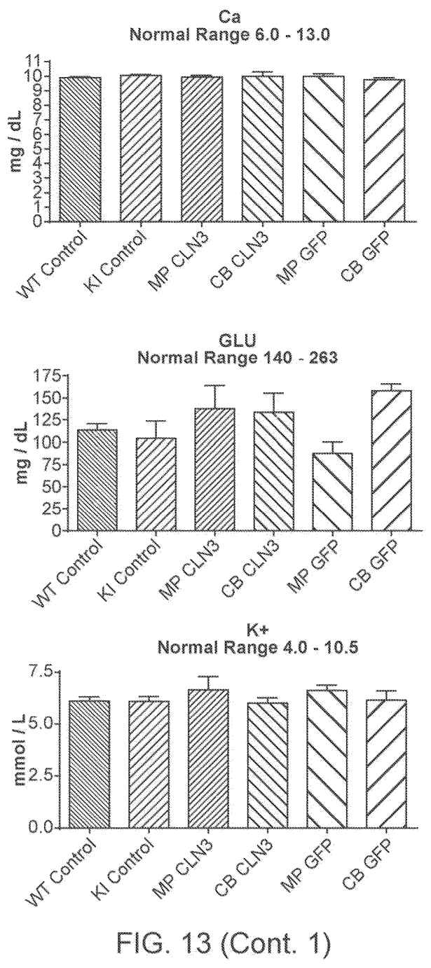

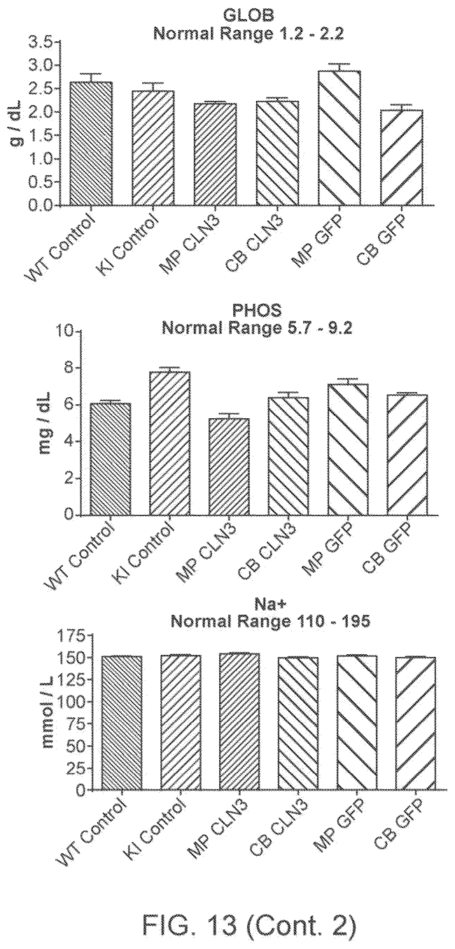

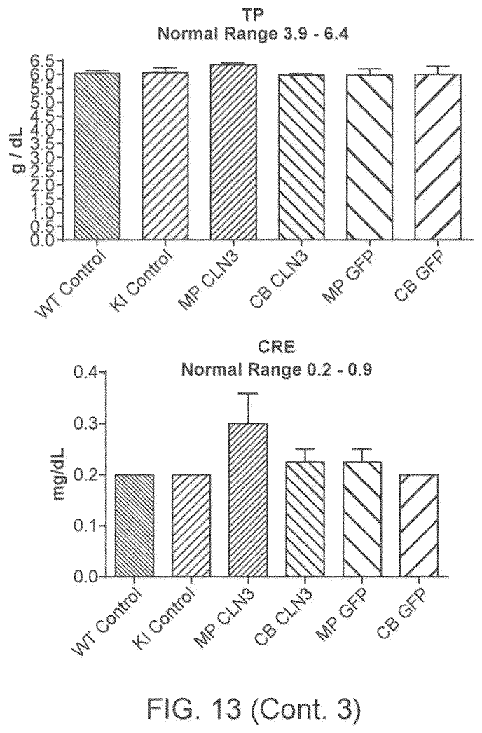

Serum chemistry panels (Abaxis, Union City, CA, USA) were run on the different experimental mouse cohorts to assess safety and toxicity of the AAV treatments. Mediators measured included albumin (ALB), alkaline phosphatase (ALP), alanine aminotransferase (ALT), amylase (AMY), bilirubin (TBIL), blood urea nitrogen (BUN), calcium (CA), phosphorus (PHOS), glucose (GLU), sodium (Na), potassium (K), total protein (TP), globulin (GLOB), and creatinine (CRE). Virus was administered to one month old mice and testing was performed at 1-month (FIG. 12), 3-months (data not shown), 5-months (data not shown), 7-months (data not shown), and 10-months (FIG. 13) post-infection. Data revealed differences in certain analyte levels, for example, increased alanine aminotransferase levels in the mice receiving AAV9/β-actin-hCLN3 as compared to the other experimental cohorts, but these were not statistically significant as to the normal ranges for each analyte.

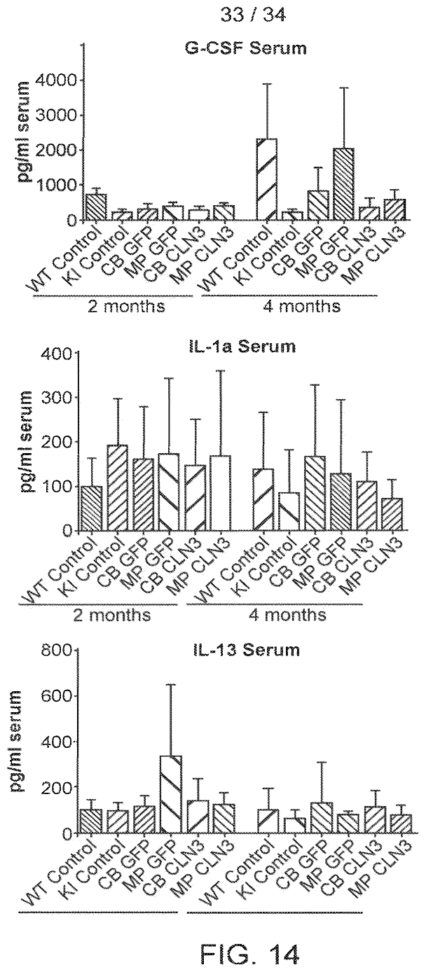

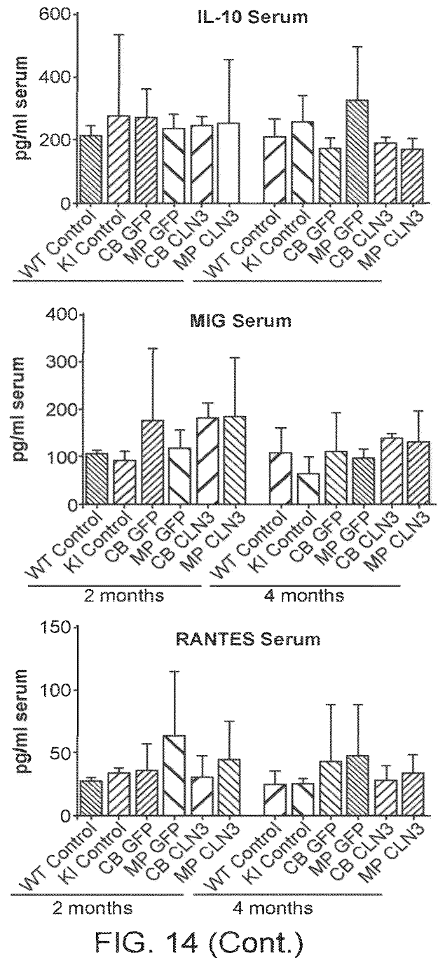

Serum inflammatory mediator analyses were also performed using MILLIPLEX® panels (EMD Millipore, Billerica, MA, USA). Mediators measured included granulocyte-colony stimulating factor (G-CSF), granulocyte-macrophage colony-stimulating factor (GM-CSF), interferon gamma (IFN-γ), interleukin-1 alpha (IL-1α), interleukin-1 beta (IL-1B), interleukin-2 (IL-2), interleukin-4 (IL-4), interleukin-5 (IL-5), interleukin-6 (IL-6), interleukin-7 (IL-7), interleukin-9 (IL-9), interleukin-10 (IL-10), interleukin-12p70 (IL-12p70), interleukin-13 (IL-13), interleukin-15 (IL-15), interleukin-17 (IL-17), chemokine (C-C) ligand 2 (CCL2), chemokine (C-C) ligand 3 (CCL3), chemokine (C-C) ligand 5 (CCL5/RANTES), chemokine (C-X-C) ligand 2 (CXCL2), chemokine (C-X-C) ligand 9 (CXCL9/MIG), chemokine (C-X-C) ligand 10 (CXCL10), tumor necrosis factor-alpha (TNF-α), and vascular endothelial growth factor (VEGF). Virus was administered to one month old mice. Samples were collected at 2- and 4-months post-transduction to assess whether AAV9 elicited evidence of systemic inflammation. Only those mediators that were detected are shown in FIG. 14. Consistent with prior studies, there was little evidence of systemic inflammation in vehicle-treated CLN3^{Δex7/8} mice as these mice typically do not display overt systemic or CNS inflammation until approximately one year of age. In addition, the AAV9 viruses did not show evidence of systemic inflammatory changes. Together these data suggest the safety of AAV9 for treatment of JNCL.

### Example 3

### AAV Manufacture

AAV9 was produced by transient transfection procedures using a double-stranded AAV2-ITR-based CB-GFP vector, with a plasmid encoding Rep2Cap9 sequence along with an adenoviral helper plasmid pHelper (Stratagene) in 293 cells. The serotype 9 sequence was verified by sequencing and identical to that previously described (Gao et al. (2002) Proc. Natl. Acad. Sci. USA, 99:11854-11859). Virus was purified by two cesium chloride density gradient purification steps, dialyzed against PBS and formulated with 0.001% Pluronic-F68 to prevent virus aggregation and stored at 4° C. All vector preparations were titered by quantitative PCR using Taq-Man technology.

The AAV construct contains two inverted terminal repeats flanking a gene expression cassette. A modification to the left inverted terminal repeat removed the site of Rep nicking thereby creating self-complementary AAV genomes. The expression cassette contains a fragment of the mouse MeCP2 promoter as previously described (Garg et al. (2013) J. Neurosci. 33:13612-13620; Adachi et al. (2005) Hum Mol Genet. 14:3709-3722) to drive transgene expression. The expression cassette contains an SV40 intron and the human CLN3 variant 2 cDNA (GenBank Accession No: NM_000086.2; SEQ ID NO: 11) followed by a bovine growth hormone polyadenylation sequence.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes, including sequence listings within.

1.-70. (canceled)

71. A composition comprising a recombinant adeno-associated viral (rAAV) vector comprising a polynucleotide sequence encoding CLN3 operably linked to a methyl-CPG binding protein 2 (MeCP2) promoter, wherein the rAAV vector is of a serotype selected from AAV1, AAV2, AAV4, AAV5, AAV6, AAV8, or AAV9.

72. The composition of claim 71, wherein the CLN3 is a human CLN3.

73. The composition of claim 71, wherein the rAAV vector comprises a polynucleotide sequence of SEQ ID NO: 2.

74. The composition of claim 71, wherein the serotype is AAV9.

75. The composition of claim 71, wherein the serotype is AAV2.

76. The composition of claim 71, wherein the rAAV vector is single stranded or self-complimentary.

77. The composition of claim 71, wherein the rAAV vector is a self-complementary AAV9 vector.

78. The composition of claim 71, wherein the rAAV vector further comprises a 5′UTR/intron selected from a SV40, a minimal SV40 intron, or CBA-MVM.

79. The composition of claim 71, wherein the rAAV vector further comprises a polyadenylation signal selected from a bovine growth hormone polyadenylation sequence, a SV40 late polyadenylation sequence, a SV40 early polyadenylation sequence.

80. The composition of claim 79, wherein the polyadenylation signal comprises a nucleic acid sequence of SEQ ID NO:3, SEQ ID NO:4, SEQ ID NO:5, or SEQ ID NO: 6.

81. The composition of claim 71, wherein the rAAV vector further comprises a posttranslational regulatory element.

82. The composition of claim 81, wherein the posttranslational regulatory element is selected from a Woodchuck Post-transcriptional Regulatory Element (WPRE), a WPRE2, or a WPRE3.

83. The composition of claim 81, wherein the posttranslational regulatory element is hepatitis B virus posttranscriptional regulatory element (HPRE).

84. The composition of claim 71, wherein the rAAV vector is a self-complementary serotype AAV9 vector comprising a polynucleotide sequence encoding CLN3 that comprises a nucleic acid sequence of SEQ ID NO: 11 operably linked to the MeCP2 promoter.

85. The composition of claim 71, wherein the rAAV vector comprises a minimal SV40 intron.

86. The composition of claim 71, wherein the composition further comprises a pharmaceutically acceptable carrier or diluent.

87. A recombinant adeno-associated viral (rAAV) vector comprising a polynucleotide sequence encoding CLN3 operably linked to a promoter selected from a methyl-CPG binding protein 2 (MeCP2) promoter, a β-glucuronidase (GUSB) promoter, a neuron-specific enolase (NSE) promoter, a synapsin promoter, an oligodendrocyte transcription factor 1 (Olig1) promoter, a chondroitin sulfate proteoglycan (Cspg4) promoter, a 2′,3′-Cyclic-nucleotide 3 '-phosphodiesterase (CNP) promoter, or a glial fibrillary acidic protein (GFAP) promoter, wherein the rAAV vector is of a serotype selected from AAV1, AAV2, AAV4, AAV5, AAV6, AAV8, or AAV9.

88. A host cell comprising the rAAV vector of claim 87.


SEQUENCE LISTING (38 sequences)

SEQ ID NO: 1 - protein, 438 aa, Homo sapiens
MGGCAGSRRRFSDSEGEETVPEPRLPLLDHQGAHWKNAVGFWLLGLCNNFSYVVMLSAAHDILSHKRTSGNQSHVDPGPTPIPHNSSSRFDCNSVSTAAVLLADILPTLVIKLLAPLGLHLLPYSPRVLVSGICAAGSFVLVAFSHSVGTSLCGVVFASISSGLGEVTFLSLTAFYPRAVISWWSSGTGGAGLLGALSYLGLTQAGLSPQQTLLSMLGIPALLLASYFLLLTSPEAQDPGGEEEAESAARQPLIRTEAPESKPGSSSSLSLRERWTVFKGLLWYIVPLVVVYFAEYFINQGLFELLFFWNTSLSHAQQYRWYQMLYQAGVFASRSSLRCCRIRFTWALALLQCLNLVFLLADVWFGFLPSIYLVFLIILYEGLLGGAAYVNTFHNIALETSDEHREFAMAATCISDTLGISLSGLLALPLHDFLCQLS

SEQ ID NO: 2 - DNA, 6055 bp, synthetic construct
  misc_feature 1..6055: note = Description of Artificial Sequence: Synthetic polynucleotide
  misc_feature 662..767: note = Mutated ITR
  misc_feature 775..1511: note = Promoter
  misc_feature 1520..1550: note = SV40 late 19s insert
  misc_feature 1520..1616: note = modSV40 late 16s insert
  misc_feature 1589..1610: note = Seq primer
  misc_feature 3052..3198: note = BGHpA
  misc_feature 3278..3418: note = ITR
  source 1..6055: mol_type = other DNA
  CDS 1683..2999: note = CLN3
gcccaatacgcaaaccgcctctccccgcgcgttggccgattcattaatgcagctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgccctgcgcgctcgctcgctcactgaggccgcccgggcaaagcccgggcgtcgggcgacctttggtcgcccggcctcagtgagcgagcgagcgcgcagagagggagtggaattctatcaaaccatctgattcaacaatgacagaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgatccaccggtatgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctgacctgcaggtctagaaagcttatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcccccccccccccccccccggcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 3-6 - skipped (4 entries)

SEQ ID NO: 7 - protein, 438 aa, Canis lupus
  REGION 1..438: note = CLN3
MGGCAGSRRRLLDSEEEETAPEPRPPRSYHKGALWKNVMGFWLLGLCNNFSYVVMLSAAHDILSHQRASGNQSHVDPDPPPTAHNSSSRFDCNSVSTAAVLLADILPTLIIKLLAPLGLHLLPYSPRVLVSGICAAGSFILVAFSHSVGTSLCGVVLASISSGVGEVTFLSLTAFYPRAVISWWSSGTGGAGLMGALSYLGLTQAGLSPQHTLLSMLGIPALMLASYFFLLTSPEPQDPGGEEEAETSARQPLIDSETPESKPDSSSNLSLQERWTVFKGLLWYIVPLVLVYFAEYFINQGLFELLFFRNTSLNHAQQYRWYQMLYQAGVFVSRSSLHCCRIRFTWVLALLQCLNLAFLLVDVWFSFLPSIYLVFLIILYEGLLGGAAYVNTFHNIALETSDQHREFAMAAACISDTLGISLSGLLALPLHDFLCHLS

SEQ ID NO: 8 - protein, 438 aa, Mus musculus
  REGION 1..438: note = CLN3
MGSSAGSWRRLEDSEREETDSEPQAPRLDSRSVLWKNAVGFWILGLCNNFSYVVMLSAAHDILKQEQASGNQSHVEPGPTPTPHNSSSRFDCNSISTAAVLLADILPTLVIKLLAPLGLHLLPYSPRVLVSGVCSAGSFVLVAFSQSVGLSLCGVVLASISSGLGEVTFLSLTAFYPSAVISWWSSGTGGAGLLGSLSYLGLTQAGLSPQHTLLSMLGIPVLLLASYFLLLTSPEPLDPGGENEAETAARQPLIGTETPESKPGASWDLSLQERWTVFKGLLWYIIPLVLVYFAEYFINQGLFELLFFRNTSLSHAQQYRWYQMLYQAGVFASRSSLQCCRIRFTWVLALLQCLNLALLLADVCLNFLPSIYLIFIIILYEGLLGGAAYVNTFHNIALETSDKHREFAMEAACISDTLGISLSGVLALPLHDFLCHLP

SEQ ID NO: 9 - protein, 438 aa, Ovis aries
  REGION 1..438: note = CLN3
MGGCAGSPRRLSDSEGEETDPAPRPPLQDSQGAHWKNAVGFWLLGLCNNFSYVVMLSAAHDILSHQRTAGNQSHVDPDPTPTSHNSSSRFDCNPVSTAAVLLADILPTLIIKLLAPLGLHLLPYSPRVLTSGICAAGSFLLVAFSHSVMISLCGVVLASISSGLGEVTFLSLTAFYPRAVISCWSSGTGGAGLLGALSYLGLTQAGLSPQHTLLSMLGIPTLLLASYFLLLTSPGPQDPRGEEDSDTAARQPLINNEASESKPGSGSPLSLQERWTVFKGLLHYIVPLVLVYFAEYFINQGLFELLFFRNTFLTHAEQYRWYQMLYQAGVFASRSSLRCCPIRHTWVLALLQCLNLAFLLVDVWLSFLPSIYLIFLIIVFEGLLGGAAYVNTFHNIALETSDEHREFAMATACISDTLGISLSGLLALPLHDFLCQLT

SEQ ID NO: 10 - protein, 438 aa, Felis catus
  REGION 1..438: note = CLN3
  VARIANT 411: note = Any amino acid
MGGCAGSRRRLLDSEGEETAPEPRPRLLDRQGALWKNAMGFWLLGLCNNFSYVVMLSAAHDILSHQRASGNQSHVDPDPAPTTHNSSSRFDCNSVSTAAVLLADILPTLVIKLLAPLGLHLLPYSPRVLVSGICSAGSFILVAFSHSVGTSLCGVVLASISSGLGEVTFLSLTAFYPRAVISWWSSGTGGAGLLGALSYLGLTQAGLSPQHTLLSMLGIPALLLASYFFLLTSPEPQDPGGEEEAETSARQPLINSEAPEAKPDSSSNLSLQERWTVFKGLLWYIVPLVLVYFAEYFINQGLFELLFFRNTSLTHAQQYRWYQMLYQAGVFVSRSSLRCCRIRFTWVLALLQCFNLAFLLVDVWLSFLPSIYLVFLIILYEGLLGGAAYVNTFHNIALETSDEHREFAMAXACISDTLGISLSGLLALPLHDFLCRLS

SEQ ID NO: 11 - DNA, 1317 bp, Homo sapiens
  misc_feature 1..1317: note = CLN3, variant 2 mRNA
  source 1..1317: mol_type = unassigned DNA
atgggaggctgtgcaggctcgcggcggcgcttttcggattccgagggggaggagaccgtcccggagccccggctccctctgttggaccatcagggcgcgcattggaagaacgcggtgggcttctggctgctgggcctttgcaacaacttctcttatgtggtgatgctgagtgccgcccacgacatccttagccacaagaggacatcgggaaaccagagccatgtggacccaggcccaacgccgatcccccacaacagctcatcacgatttgactgcaactctgtctctacggctgctgtgctcctggcggacatcctccccacactcgtcatcaaattgttggctcctcttggccttcacctgctgccctacagcccccgggttctcgtcagtgggatttgtgctgctggaagcttcgtcctggttgccttttctcattctgtggggaccagcctgtgtggtgtggtcttcgctagcatctcatcaggccttggggaggtcaccttcctctccctcactgccttctaccccagggccgtgatctcctggtggtcctcagggactgggggagctgggctgctgggggccctgtcctacctgggcctcacccaggccggcctctcccctcagcagaccctgctgtccatgctgggtatccctgccctgctgctggccagctatttcttgttgctcacatctcctgaggcccaggaccctggaggggaagaagaagcagagagcgcagcccggcagcccctcataagaaccgaggccccggagtcgaagccaggctccagctccagcctctcccttcgggaaaggtggacagtgttcaagggtctgctgtggtacattgttcccttggtcgtagtttactttgccgagtatttcattaaccagggactttttgaactcctctttttctggaacacttccctgagtcacgctcagcaataccgctggtaccagatgctgtaccaggctggcgtctttgcctcccgctcttctctccgctgctgtcgcatccgtttcacctgggccctggccctgctgcagtgcctcaacctggtgttcctgctggcagacgtgtggttcggctttctgccaagcatctacctcgtcttcctgatcattctgtatgaggggctcctgggaggcgcagcctacgtgaacaccttccacaacatcgccctggagaccagtgatgagcaccgggagtttgcaatggcggccacctgcatctctgacacactggggatctccctgtcggggctcctggctttgcctctgcatgacttcctctgccagctctcctga

SEQ ID NO: 12 - protein, 735 aa, synthetic construct [sequence omitted]

SEQ ID NO: 13 - protein, 745 aa, synthetic construct
  REGION 1..745: note = Description of Artificial Sequence: Synthetic AAV9 VP1 sequence
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQGQRGNYSRGVDAQAAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 598 aa, synthetic construct
  REGION 1..598: note = Description of Artificial Sequence: Synthetic AAV2 VP2 sequence
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 15 - protein, 518 aa, synthetic construct
  REGION 1..518: note = Description of Artificial Sequence: Synthetic AAV9 VP2 sequence
DGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 533 aa, synthetic construct
  REGION 1..533: note = Description of Artificial Sequence: Synthetic AAV2 VP3 sequence
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 17 - protein, 533 aa, synthetic construct; identical to SEQ ID NO: 16
  REGION 1..533: note = Description of Artificial Sequence: Synthetic AAV9 VP3 sequence

SEQ ID NO: 18 - protein, 621 aa, synthetic construct
  REGION 1..621: note = Description of Artificial Sequence: Synthetic AAV2 Rep78 sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 19 - protein, 536 aa, synthetic construct
  REGION 1..536: note = Description of Artificial Sequence: Synthetic AAV2 Rep68 sequence
MPGFYEIVIKVPSDLDEHLPGISDSFVNWVAEKEWELPPDSDMDLNLIEQAPLTVAEKLQRDFLTEWRRVSKAPEALFFVQFEKGESYFHMHVLVETTGVKSMVLGRFLSQIREKLIQRIYRGIEPTLPNWFAVTKTRNGAGGGNKVVDECYIPNYLLPKTQPELQWAWTNMEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 20 - protein, 397 aa, synthetic construct
  REGION 1..397: note = Description of Artificial Sequence: Synthetic AAV2 Rep52 sequence
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 21 - protein, 312 aa, synthetic construct
  REGION 1..312: note = Description of Artificial Sequence: Synthetic AAV2 Rep40 sequence
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRLARGHSL

SEQ ID NO: 22 - DNA, 546 bp, Mus musculus
  misc_feature 1..546: note = MeCP2 Variable Promoter Sequence
  source 1..546: mol_type = unassigned DNA
gaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 23 - DNA, 737 bp, Mus musculus
  misc_feature 1..737: note = MeCP2 Variable Promoter Sequence
  source 1..737: mol_type = unassigned DNA
tcaaaccatctgattcaacaatgcacgaccgatctcttatgggcttggcacacaccatctgcccattataaacgtctgcaaagaccaaggtttgatatgttgattttactgtcagccttaagagtgcgacatctgctaatttagtgtaataatacaatcagtagaccctttaaaacaagtcccttggcttggaacaacgccaggctcctcaacaggcaactttgctacttctacagaaaatgataataaagaaatgctggtgaagtcaaatgcttatcacaatggtgaactactcagcagggaggctctaataggcgccaagagcctagacttccttaagcgccagagtccacaagggcccagttaatcctcaacattcaaatgctgcccacaaaaccagcccctctgtgccctagccgcctcttttttccaagtgacagtagaactccaccaatccgcagctgaatggggtccgcctcttttccctgcctaaacagacaggaactcctgccaattgagggcgtcaccgctaaggctccgccccagcctgggctccacaaccaatgaagggtaatctcgacaaagagcaaggggtggggcgcgggcgcgcaggtgcagcagcacacaggctggtcgggagggcggggcgcgacgtctgccgtgcggggtcccggcatcggttgcgcgcgcgctccctcctctcggagagagggctgtggtaaaacccgtccggaaaa

SEQ ID NO: 24 - DNA, 556 bp, synthetic construct
  misc_feature 1..556: note = Description of Artificial Sequence: Synthetic CBA promoter with CMV enhancer sequence
  source 1..556: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctactcgaggccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggag

SEQ ID NO: 25 - DNA, 2280 bp, synthetic construct
  misc_feature 1..2280: note = Description of Artificial Sequence: Synthetic Neurofilament light-chain gene promoter sequence
  source 1..2280: mol_type = other DNA
gtaaatactaagagtgttttatgcagactcacacacactaagtgtggaacaatgaggtttgcagggagcaggttaacaaaataatccattctgccatgcttcatctacatatgggtaattgggaggagacaactctatggcttatctgggtccttgttccaccctccgcctccagagaagcctattccacccttgaatttcatgagtgaaaagaccaaggatagctaggctatgaaaagtcaacagagtaagtaccctaccaaacctgccagggacaaacaatattcaggacagcctcatgaccttaggcttaaaatctacaatatttgggcatggatggtcctctgagatcacttagtcttgatagatttaaactactaattttcaatatttgtgaatttgcattatttgcgacaacagacattgtttagaaacctgaggaaagttatctgctgcagtctatcatgaggacggtgggctatcacaatcacaggtattttttttggggggggggatgagaaagggcctctgaagataagcctcaccccatcctttcctcgggagagagtgaaatgtctccagaagcaactgcaccactgcaaatacttcaccctccaagtatcacaggggaagccattccgggcttctctatttgaaaacaattaccatattccccctcagtttgcctcttaaaaaaaacataagttgcacaatcaaatgctgcgatatacaaataaaacaaaaactaaatgcttgctcagataaatcttaggtgtttcgtgtaaatctctgcaatccctccatgaaacctgggggactcctaacatcatgcgtgtgaaaatgccctgcaaaccgtagggttgtatccacgtgcgctaccgcgtgcgccagtttcaagcatctgggtgcttcctgaaggaaatccatgcattcctgactgcatctgttttcgggttattacattgtatcggggaagagtcacgcggctatggctattttctatccgttcctgaagaagcctgggtgccgagttctcctccccagagaccacacccagcgcttagggctggccgcagcggcttccctgaaaatcagccaactgcaaggcttatcgaaatcatcaggtcgtgtgctacacatgtgtaaaaagaggaaagcggactttaaatgtgctgcggttggtggtagcaagcaggaatttagcttggtgaggatccaggcagcttgaagctcccggctgcggacgcgcggctccctcagcaagtcagtctctgtgtttccaactctttctctgccctcaccacccacacactgcaacacgttaaagccatctgcggcttcattctcagttaagaaatgtgaaaatctagaaacactaacaggcggattaactgctgtaagggtttaaaaatgctaaaccaatacctgcagtagtgccgcagtttcacgagtgtgtgtgtgtgtgtgtgtgtgcgcgcgcgcgcatcgcgcgacactccctatgtgttaagcagctcattaaagaaaaagaaaaataatcaggagaaaggaagatgaattacagaaagtgccagaaagctagaaagaaattaaaactcttctccatacatactgcatacacataacctagcctatttatttgtatctaaaattccctagccgcaccatcaccgtaaacaccaagggaaaaaattaaggaggttcctggtgggaaaagggcgagttggggggacagggtgtctgcgaggtgacgggatacacaaaactagggtgtcaaaagggagcaagaacctgttttgagggcaacttaaggatccaagtgtcacggggtctgggcaatgaaggacgggaggggctgcgtgagtgagtacagaagggaaatgagtgagggggcatgggatctcagagaaaatcagggccctctgagcaaagtggaaaggacgaccgccgcagctcctcgggccgtagcccgaccccgccttcccttttgcgcagaatcctcgccttggctgcagcagcgcgctgcccccactggccggcgtgccgtgatcgatcgcaggctgcgtcaggacctcccggcgtataaataggggtggcagaacggcgccgagccgcacacaaccatccatcctcccccttccctctctcccctgtcctctctctccgggctcccaccgccgccgcgggccggggagcaccggccgccaccatgagttccttcagctacgagccgtactactcgacctcctacaagcggcgctacgtggagacgccccgggt

SEQ ID NO: 26 - DNA, 2356 bp, synthetic construct
  misc_feature 1..2356: note = Description of Artificial Sequence: Synthetic Neuron-specific vgf gene promoter sequence
  source 1..2356: mol_type = other DNA
ctcgaggatctgattaaatacacaattgcttccccccattccctttctttttctccccgccccggccatgtatctcattcatctccatacacacataaacacacatgcacaagccatgtacatgtacacgcaggtgtgtgtgcatacacaagccaacaggcaaatacagtttctccaggtgcctgtcttctctcatcttgcaacttggtctctgatccccatcagccactcagtcagcccccttggctccctccctcccctctcccttctctcttggatgggttcccctccccctctccagatgtctgagccatcttctctctgattcatcctcctcaggaaggaacgtgaccccctccccatcccactgcctctgtatcaggctgggaagatgaaggggacatgggggcggggagaggaaggaggggaggccgtggttagttgtgcgtggggatgggaggcattgcctggggtctcctaccccctcttttcccctccctttctttggaatctccactgtcaccttggttctcagtttttttttctcctttagcctgctccttctacctgttccagatcccttcattccttcctcctcccctgcccccatctcttctctcttttctccctctccactcctccccatttctttcccgccaagagctgatgggctttcttctgggaaagtcgagccactgatggaagcgagaagccactgctggttatagagagaaagcacgtgagtgtgtgtgtagggagggggaggttagaaggagggtcagtgccaggaagaggtgaggaggggggggaggaccgtttctgaaagagtctctaagaccctgacagacagccctgaccttggtttccagagtctcagggtgcggtgccctgcgtgtgcccacagagcacccctatgtccgcagttcgtgtgtgtctggcgtgtgtcattgtattcccccccccttgggtgcccaggcccgccaccgctctctgccagcaccgcagccccctccaggcttcctccctccctccccttcattcctgcagtggctgccccccttgccaccctctcctctcccctgccccctccccatttcctgcctcccccccacccgccccacggctggtctcccttgaccggacccagctctctgatggattctctttgcgcaaatctgtgcgtcatcgcccccacccccggaacctctagctgtccaagcccccagccccaacctctctggcaggagatacggtcgaaggggctggtggcagagaggggctatctctgacgttgcaggtccccctcccatcgcgttcaaaccttccctttaagcggtggagagagctggagttgagtcacccccccccccacctgcgcaaccccctccccacctgctctggtctcgccctccaaacgtccttgggggaggggagcgggccaggagggaaagcgactggggagtgtgggaagagatggggccgaagggggcacagcggggggccttgacacaagcggcagtcaggggacagaaggacagacacacctttttctccagacacagcacggatcgtgaaacagacacgacccagaggcacacacatcctcattctttcccttttctcttccgactcggacccttccgatgggattaccaaaaccgcaagatccacccatctccgctgtcaggggctgcaccccgactgcccattccgggacagccgcaggcgtgcagatctgtccctctgcactcaggttcacgccgtccttggggccgtggtctcggggtggggaaccggcccctggtcggctcttgaatctttatccttcccctccccagtattgagctcccactggtgcccagtcagacgctgggactaccctttttctattccactcagcaacgcgggctccatccagcagctccaagttgctctgcaacccaccctcccgccttccagcgcctctgcatccacccttccattcattctcccattcattcattcatccttttctcctcgtccctccttcattcattcatagccccccgccctgcccgcttcagcatttcattcattcattcattcattcatttcccggagctccgctagcgcacaccccttcagccgaagccccagcgcgcaggcgcaggccgggagaggcaggcaccctccaatcgtcgggcgtccttcctcctccgggcggccgcccgcttccccatgaatgaacattgacgtcaatggggcggggcgcgcccacgtgaccccgcgcgctcccctttataaggcggtggaggcgcggggctgtccagcgtgctgaagcggagcgagctagccgcccggagccgcgccgacccag

SEQ ID NO: 27 - DNA, 576 bp, synthetic construct
  misc_feature 1..576: note = Description of Artificial Sequence: Synthetic CMV promoter sequence
  source 1..576: mol_type = other DNA
tagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcag

SEQ ID NO: 28 - DNA, 454 bp, synthetic construct
  misc_feature 1..454: note = Description of Artificial Sequence: Synthetic RSV promoter sequence
  source 1..454: mol_type = other DNA
cgacaattgcatgaagaatctgcttagggttaggcgttttgcgctgcttcgcgatgtacgggccagatatacgcgtatctgaggggactagggtgtgtttaggcgaaaagcggggcttcggttgtacgcggttaggagtcccctcaggatatagtagtttcgcttttgcatagggagggggaaatgtagtcttatgcaatacacttgtagtcttgcaacatggtaacgatgagttagcaacatgccttacaaggagagaaaaagcaccgtgcatgccgattggtggaagtaaggtggtacgatcgtgccttattaggaaggcaacagacaggtctgacatggattggacgaaccactgaattgcgcattgcagagataattgtatttaagtgcctagctcgatacaataaacgccatttgaccattcaccacattggtgtgcacctccaaggcc

SEQ ID NO: 29 - DNA, 350 bp, synthetic construct
  misc_feature 1..350: note = Description of Artificial Sequence: Synthetic SV40 promoter sequence
  source 1..350: mol_type = other DNA
cagctgtggaatgtgtgtcagttagggtgtggaaagtccccaggctccccagcaggcagaagtatgcaaagcatgcatctcaattagtcagcaaccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctttgcaaagatggataaagtt

SEQ ID NO: 30 - DNA, 511 bp, synthetic construct
  misc_feature 1..511: note = Description of Artificial Sequence: Synthetic PGK promoter sequence
  source 1..511: mol_type = other DNA
gatctctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctacccctcccctagtcaggaagttcccccccgccccgcagctcgcgtcatgcaggacgtgacaaatggaagtagcacgtctcactagtctcgtgcaaatggacagcaccgctgagcaatggaagcgggtaggcccttggggcagcggccaatagcagctttgctccttcgctttctgggctcagaggctgggaaggggtgggtccgggggcgggctcaggggcgggctcaggggcggggcgggcgcccgaaggtcctccggaggcccggcattccgcacgcttcaaaagcgcacgtctgccgcgctgttctcttcttcctcatctccgggcctttcga

SEQ ID NO: 31 - DNA, 1211 bp, synthetic construct
  misc_feature 1..1211: note = Description of Artificial Sequence: Synthetic EFalpha promoter sequence
  source 1..1211: mol_type = other DNA
gatctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcactagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgtggccttgcgcttaaggagccccttcgcctcgtgcttgagttgtggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcagcacactggtatttcggtttttggggccgcgggcggcgacggggcccgtgcgtcccagcgcacatgttcggcgaggcggggcctgcgagcgcggccaccgagaatcggacgggggtagtctcaagctgcccggcctgctctggtgcctggcctcgcgccgccgtgtatcgccccgccctgggcggcaaggctggcccggtcggcaccagttgcgtgagcggaaagatggccgcttcccggccctgctgcagggagcacaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctccagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaactacccctaaaagccaaaa

SEQ ID NO: 32 - DNA, 408 bp, synthetic construct
  misc_feature 1..408: note = Description of Artificial Sequence: Synthetic Synapsin promoter sequence
  source 1..408: mol_type = other DNA
agatctctgcagaaggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcga

SEQ ID NO: 33 - DNA, 336 bp, synthetic construct
  misc_feature 1..336: note = Description of Artificial Sequence: Synthetic NSE promoter sequence
  source 1..336: mol_type = other DNA
atggcgccgggcctttctttatgtttttggcgtcttccatggtggctttaccaacagtaccggaatgccaagcttacttagatcgcagatctcggtggtagtggcggtggcggtggcggtggcgacggcggcgctgagagagcgggagtggcagtggcggcggcggctgcggctcccgggcggcgggcggaggaggcgcctatagggccgcgcgggcgcatgtgacccggagcccccgatgagtcaggagctgccggcggaggcgcacgtacgagcgcgggtgggggaccgacgagggtggagtggggaagggaggaggatgggggaagggtgggg

SEQ ID NO: 34 - DNA, 1219 bp, synthetic construct
  misc_feature 1..1219: note = Description of Artificial Sequence: Synthetic UBC promoter sequence
  source 1..1219: mol_type = other DNA
gatctggcctccgcgccgggttttggcgcctcccgcgggcgcccccctcgtcacggcgagcgctgccacgtcagacgaagggcgcaggagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgagcccttcgctaatgcgggaaagctcttattcgggtgagatgggctgggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagacttgtaaattgtccgctaaattctggccgtttttggcttttttgttagacaaca

SEQ ID NO: 35 - protein, 738 aa, Adeno-associated virus
  REGION 1..738: note = AAV 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSGPRPIGTRYLTRNL

SEQ ID NO: 36 - DNA, 1877 bp, Adeno-associated virus
  misc_feature 1..1877: note = AAV 9
  source 1..1877: mol_type = unassigned DNA
ctggttgaggaaggcgctaagacggctcctggaaagaagagaccggtagagcagtcaccccaagaaccagactcatcctcgggcatcggcaaatcaggccagcagcccgctaaaaagagactcaattttggtcagactggcgactcagagtcagtccccgacccacaacctctcggagaacctccagaagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggcattgcccacctacaacaaccacctctacaagcaaatctccaatggaacatcgggaggaagcaccaacgacaacacctactttggctacagcaccccctgggggtattttgacttcaacagattccactgccacttctcaccacgtgactggcagcgactcatcaacaacaactggggattccggccaaagagactcaacttcaagctgttcaacatccaggtcaaggaggttacgacgaacgaaggcaccaagaccatcgccaataaccttaccagcaccgtccaggtctttacggactcggagtaccagctaccgtacgtcctaggctctgcccaccaaggatgcctgccaccgtttcctgcagacgtcttcatggttcctcagtacggctacctgacgctcaacaatggaagtcaagcgttaggacgttcttctttctactgtctggaatacttcccttctcagatgctgagaaccggcaacaactttcagttcagctacactttcgaggacgtgcctttccacagcagctacgcacacagccagagtctagatcgactgatgaaccccctcatcgaccagtacctatactacctggtcagaacacagacaactggaactgggggaactcaaactttggcattcagccaagcaggccctagctcaatggccaatcaggctagaaactgggtacccgggccttgctaccgtcagcagcgcgtctccacaaccaccaaccaaaataacaacagcaactttgcgtggacgggagctgctaaattcaagctgaacgggagagactcgctaatgaatcctggcgtggctatggcatcgcacaaagacgacgaggaccgcttctttccatcaagtggcgttctcatatttggcaagcaaggagccgggaacgatggagtcgactacagccaggtgctgattacagatgaggaagaaattaaagccaccaaccctgtagccacagaggaatacggagcagtggccatcaacaaccaggccgctaacacgcaggcgcaaactggacttgtgcataaccagggagttattcctggtatggtctggcagaaccgggacgtgtacctgcagggccctatttgggctaaaatacctcacacagatggcaactttcacccgtctcctctgatgggtggatttggactgaaacacccacctccacagattctaattaaaaatacaccagtgccggcagatcctcctcttaccttcaatcaagccaagctgaactctttcatcacgcagtacagcacgggacaagtcagcgtggaaatcgagtgggagctgcagaaagaaaacagcaagcgctggaatccagagatccagtatacttcaaactactacaaatctacaaatgtggactttgctgtcaataccgaaggtgtttactctgagcctcgccccattggtactcgttacctcacccgtaatttgtaattgcctgttaatcaataaaccggttaattcgtttcagttgaactttggtctctgcg

SEQ ID NO: 37 - DNA, 597 bp, synthetic construct
  misc_feature 1..597: note = Description of Artificial Sequence: Synthetic WPRE enhancer sequence
  source 1..597: mol_type = other DNA
cgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcg

SEQ ID NO: 38 - DNA, 606 bp, synthetic construct
  misc_feature 1..606: note = Description of Artificial Sequence: Synthetic WPRE enhancer sequence
  source 1..606: mol_type = other DNA
cgactgatccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcg